(19) United States
(12) Patent Application Publication
(10) Pub. No.: US 2005/0058641 A1
(43) Pub. Date: Mar. 17, 2005

(54) Tolerance induction and maintenance in hematopoietic stem cell allografts

(76) Inventor: Maria Z. Siemionow, Cleveland, OH (US)

Correspondence Address: JONES DAY, 222 EAST 41ST ST, NEW YORK, NY, 10017, US

(21) Appl. No.: 10/852,936

(22) Filed: May 24, 2004

Related U.S. Application Data
(63) Continuation-in-part of application No. 10/443,281, filed on May 22, 2003.
(60) Provisional application No. 60/472,553, filed on May 22, 2003.
(60) Provisional application No. 60/382,680, filed on May 22, 2002.

(51) Int. Cl. A61K 48/00; A61K 39/395

(52) U.S. Cl. 424/144.1; 424/93.21; 424/93.7

(57) ABSTRACT

Provided are methods and compositions for the induction and maintenance of tolerance in hematopoietic stem cell allografts.

This application claims the benefit of U.S. Provisional Application No. 60/472,553, filed May 22, 2003, and is a continuation-in part of U.S. patent application Ser. No. 10/443,281, filed May 22, 2003, which claims benefit of U.S. Provisional Application No. 60/382,680, filed May 22, 2002, the entire disclosures of which are hereby incorporated by reference herein.

### 1. FIELD OF THE INVENTION

The present invention relates to the field of immunology and the field of transplantation. Specifically, the invention relates to the field of organ, composite tissue allograft, and hematopoietic stem cell transplantation.

### 2. BACKGROUND OF THE INVENTION

Composite tissue allografts (CTAs) are neurovascularized allografts of tissues that include structural, functional, and aesthetic units of integumentary and musculoskeletal elements. Although not vital to life, CTAs are important to those who deal with the functional restoration of musculoskeletal defects. CTAs are unique in that they are a heterogeneous histological milieu of tissue elements, with each component possessing different antigen expression and presentation mechanisms. For example, CTAs can be comprised of several tissue types including skin, subcutaneous tissue, nerve and vascular tissues, bone, muscle, fascia, cartilage, and the like. In addition, CTAs can contain immunocompetent elements, such as bone marrow and lymph nodes, that can hasten the graft rejection processes and/or result in graft versus host disease (GVHD). The heterogeneous nature of CTAs not only affects the immune reactivity of these allogeneic tissues, but also defines potential immunomodulating strategies that may be different from those currently used in solid organ transplantation. That the majority of attempts at CTA transplantation have been unsuccessful illustrates the difficult barrier associated with a neurovascularized allograft composed of a variety of tissues.

Stem cell transplantation is a promising new strategy for the therapy of many human disorders. Hematopoietic stem cell transplantation in particular has significant potential for the treatment of malignancy, autoimmunity and genetic disorders. It can also be used to facilitate gene therapy and solid organ, as well as composite tissue allograft transplantation. However, current protocols for allogeneic hematopoietic stem cell transplantation suffer from prohibitive morbidity and mortality due to the toxic effects of the conditioning regimens they require, such as immunosuppressant and myeloablation, and because of the occurrence of graft-versus-host disease (GVHD). Both the conditioning regimen and immunosuppressive therapy to prevent or treat GVHD pose risks of infection and malignancy. Currently, allogeneic hematopoietic stem cell transplantation requires substantial cytoreductive treatment of recipients with irradiation or cytotoxic drugs to achieve adequate levels of stable donor engraftment. The multiorgan systemic toxicity associated with conditioning and the high incidence of complications have limited the practical clinical applicability of allogeneic hematopoietic stem cell transplantation. A method of hematopoietic stem cell transplantation that works with a nonmyeloablative, nontoxic conditioning regimen and results in durable donor chimerism in the recipient without triggering GVHD would thus be a significant advance in the treatment of hematologic disorders, autoimmune diseases, and for the induction of tolerance for solid organ, as well as composite tissue allograft transplantation.

Thus, there exists a need in the art for relatively non-toxic, noninvasive methods of transplanting complex tissues and stem cells that encourage long-term survival of the transplants. The present invention provides such methods.

### 3. SUMMARY OF THE INVENTION

The present invention provides a new approach for inducing long-term, donor-specific tolerance to donor antigens, especially in recipients of CTA transplants although not limited thereto, without the requirement for recipient conditioning, without the need for chronic immunosuppressive regimens, and without the occurrence of GVHD. The methods according to the invention are fully applicable to transplantation of any type of allograft including, but not limited to, composite tissue such as, but not limited to human hand, human finger, human larynx, joints such as knee, hip, limbs, lower extremities, and the like; solid organs and glands such as, but not limited to, heart, lung, kidney, liver, pancreas, thyroid, and the like; glandular cells such as, but not limited to, islet cells and the like; skin; cartilage, such as an ear; hematopoietic tissue; lymphoid tissue; tendons; ligaments; muscles; nerve tissue vascular tissue such as vessels; and the like, without limitation.

In one embodiment of the invention, a method is provided for inducing donor-specific tolerance and/or mixed donor-recipient chimerism in an allograft transplant recipient, comprising administering to a recipient of an allograft a therapeutically effective amount of an immunosuppressive agent that depletes T cells; administering to the recipient of the allograft a therapeutically effective amount of anti-T cell receptor antibodies; implanting an allograft from an allograft donor into the recipient; and administering to the recipient a therapeutically effective amount of hematopoietic stem cells from the allograft donor by intraosseous delivery. In various, embodiments intraosseous delivery is accomplished by direct injection; implantation of bone marrow, unprocessed or processed; transplantation of a composite tissue allograft containing bone marrow; transplantation of stem, progenitor or other donor bone marrow-derived cells; transplantation of an artificial or commercially-available matrix seeded with donor bone marrow-derived cells, etc.

The immunosuppressive agent employed in the embodiments of the invention is preferably an inhibitor of the calcineurin pathway of T cell activation such as, but not limited to, cyclosporine A (CsA), FK-506, and the like; and/or other inhibitors of IL-2 production such as, but not limited to, rapamycin and the like, and combinations of the foregoing. More preferably, the immunosuppressive agent is CsA.

In a preferred embodiment, the immunosuppressive agent and the anti-αβ T cell receptor antibodies are administered as a short course of therapy that can be initiated prior to transplantation, alternatively at transplantation, alternatively about one to about three days after transplantation. Preferably, the therapy continues for a short time period after transplantation. In specific embodiments, for example, administration of the immunosuppressant and the anti-αβ T cell antibodies may continue for 1, 2, 3, 4, 5, 7, 10 or 20 days after transplantation. In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention.

The donor hematopoietic stem cells are preferably administered at the time of transplantation, but can be administered at any time up to about three days after transplantation. The embodiments of the invention also encompass readministering of donor hematopoietic stem cells at any time post-transplant, particularly if the level of chimerism in the recipient declines and/or at the onset of allograft rejection.

The effective amount of allograft donor hematopoietic stem cells is preferably an amount sufficient to induce the production of mixed donor-recipient chimeric cells in the allograft recipient and, preferably, is an amount sufficient to maintain long-term recipient tolerance of the allograft without the necessity of readministration of the immunosuppressive agent and the anti-T cell receptor antibodies.

In another embodiment, a combination of the steps of administering the immunosuppressive agent and the anti-T cell receptor antibodies, and administering the donor hematopoietic stem cells, results in induction of hematopoietic mixed donor-recipient chimerism and/or long-term allograft tolerance in the recipient.

In some embodiments, methods of administering the donor hematopoietic stem cells into the recipient include, but are not limited to, implantation and vascularization of a donor bone containing bone marrow, intraosseous delivery of crude donor bone marrow, intraosseous delivery of a suspension of nucleated donor bone marrow cells, intraosseous delivery of a suspension of a nucleated cell subpopulation isolated from donor bone marrow cells, and the like.

In other embodiments, isolated subpopulations of the donor bone marrow nucleated cells such as, but not limited to, tolerance inducing cells, hematopoietic stem cells, hematopoietic progenitor cells, CD4⁺ cells, CD8⁺ cells, donor-derived B-cells, and combinations of the foregoing, are administered to the recipient by intraosseous delivery. Such subpopulations can be isolated by methods well known in the art. In a further embodiment, a method is provided for inducing donor-specific tolerance and/or mixed donor-recipient trimerism in an allograft transplant recipient, comprising the steps of: (a) administering to a recipient of an allograft a therapeutically effective amount of an immunosuppressive agent that depletes T cells; (b) administering to the recipient of the allograft a therapeutically effective amount of anti-T cell receptor antibodies; (c) implanting a first allograft from a first allogeneic allograft donor into the recipient; (d) administering a therapeutically effective amount of hematopoietic stem cells from the first allograft donor into the recipient; (e) implanting a second allograft from a second allogeneic allograft donor into the recipient; and (e) administering a therapeutically effective amount of hematopoietic stem cells from the second allograft donor into the allograft recipient. In still a further embodiment, the method can optionally include the steps of implanting additional allografts and hematopoietic stem cells from additional donors, to induce tolerance to multiple donor allografts and/or mixed donor-recipient multimerism in the transplant recipient.

In further embodiments of the invention, methods are provided for monitoring allograft rejection in an allograft transplant recipient by implanting an additional allograft from the donor into the recipient. Preferably, the additional allograft can exhibit visible or histological signs that are readily ascertainable, and can indicate early recipient allograft rejection. As a non-limiting example, the additional allograft can include vascularized and/or non-vascularized skin; artificial skin, where the artificial skin is preferably implanted with donor keratinocytes, preferably non-allogenic, and the like. For example, a donor bone for implantation and vascularization in the recipient can include an attached portion of donor skin that also can be implanted and vascularized into the recipient as a monitor of allograft rejection. The additional, monitoring allograft can be especially useful as an indicator for monitoring graft rejection when the transplant recipient has received an internal allograft such as, but not limited to, a solid organ, or the like.

In yet other embodiments of the inventions, methods are provided for maintaining a level of mixed donor-recipient chimerism in an allograft transplant recipient. In one of these embodiments, the method comprises inducing donor-specific tolerance and/or mixed donor-recipient chimerism in an allograft recipient by any embodiment of the above-described methods, and maintaining a desired chimerism level by determining an optimal level of chimeric cells in the recipient not undergoing rejection of the allograft; harvesting chimeric cells from the recipient when an optimal level of chimeric cells is achieved; reconstituting the recipient with the harvested chimeric cells when the level of chimeric cells falls below a minimum level of chimeric cells; and, optionally, readministering an effective amount of the immunosuppressive agent and/or the anti-T cell receptor antibodies sufficient to restore the desired chimerism level. In another embodiment, where chimeric cells, exhibiting a particular, desired level of chimerism, have been harvested from a recipient and stored (for example, in a chimeric cell bank), a particular level of chimerism may be maintained by obtaining all or a portion of the stored cells an administering them to the recipient. Such transplantations may take place a plurality of times after the original transplantation.

Embodiments of the invention include a system for inducing donor-specific tolerance and/or mixed donor-recipient chimerism in an allograft transplant recipient, comprising (a) a combination of pharmaceutical compositions for depletion of T cells in a recipient of an allograft from a donor, comprising an effective amount of a pharmaceutical composition that comprises an immunosuppressive T cell-depleting agent, and an effective amount of a pharmaceutical composition that comprises anti-αβ TCR⁺ T cell receptor antibodies, wherein administration of the combination to the recipient results in elimination of about 50% to about 99.9% of T cells circulating in peripheral blood of the recipient; and (b) a delivery system for administering a therapeutically effective amount of hematopoietic stem cells from the allograft donor into the allograft recipient by intraosseous delivery, wherein the delivery system is selected from the group consisting of an implantable vascularizable bone from the allograft including the donor bone marrow; implantable crude donor bone marrow; an implantable donor bone marrow cell suspension; an implantable isolated subpopulation of nucleated donor bone marrow cells; implantable commercially-available scaffold; and combinations of the foregoing.

The embodiments of the system and methods according to the invention are fully clinically applicable to transplantation in human recipients and, for example, are adaptable to take into account such uncertainties as the timing of the availability of allograft transplants for human recipients, and the like. Embodiments according to the invention are applicable to semi-allogeneic transplants such as, but not limited to, transplantation between related donor/recipients that are partially-mismatched at a major histocompatibility complex (MHC) class I or class II locus, and to fully-allogeneic transplants such as, but not limited to, transplantation between unrelated, fully mismatched MHC donor/recipient, including xenogeneic transplants to humans. In specific embodiments, transplants according to the invention are performed wherein the donor and recipient share no histocompatibility loci, for example, transplants between species, or transplants between donor and recipient of the same species wherein donor and recipient share no loci. In a more specific example, the transplant is from a human donor to a human recipient, where the donor and recipient share no HLA markers. The invention encompasses the transplantation from donors to recipients of any species, wherein the donor and recipient share no immunodeterminants analogous to HLA markers or MHC loci.

In the embodiments of the invention, the donor can be a mammal of a first species and the recipient can be a mammal of a second species. In further embodiments, the donor and the recipient can be of the same species. In other embodiments, donor and recipient are mammals, birds, reptiles, amphibians or marsupials. In a specific embodiment, said mammals are domestic mammals. In a more specific embodiment, said domestic mammal is a canine or a feline. In another more specific embodiment, said domestic mammal is an equine, bovine, porcine species. In yet further embodiments, the recipient is a primate. In a preferred embodiment, the recipient is a human.

In various embodiments, donor and recipient may be genetically unrelated individuals, or may be from the same immediate family. In other embodiments, donor and recipient may share 1 or more HLA markers, or other immune-determining markers, or may share none.

In various embodiments, the anti-αβ TCR receptor antibodies used in the methods of the present invention may be derived from cells of the same species as the intended recipient (for example, human anti-αβ TCR receptor antibodies may be administered to a human recipient). In other embodiment, the antibodies are modified for administration to a particular species. In specific embodiments, for example, the antibody may be a mouse-anti-rat T cell receptor antibody; a humanized rabbit antibody; etc. In embodiments employing human recipients, the anti-αβ TCR receptor antibodies preferably comprise human antibodies to human αβ TCR⁺ T cells. The anti-αβ TCR receptor antibodies are preferably monoclonal antibodies which can be humanized antibodies, but are preferably fully human polyclonal or monoclonal antibodies to human anti-αβ TCR receptors. In another preferred embodiment, the antibody is a murine antibody. In another embodiment, the antibody is a mouse anti-rat antibody. In a specific example, the mouse anti-rat antibody is antibody R73. In another embodiment, the anti-T cell receptor antibody is the mouse anti-human antibody designated MEDI-500 (T10B9-1A-31; Medimmune). In other embodiments, the anti-T cell receptor antibody is a recombinant antibody. In a specific example, the recombinant antibody is produced by a plant. In an even more specific example, the plant-produced antibody is produced by a corn plant.

As used herein, the terms “chimera” or “chimerism” denote a state in which cells from a donor and cells from a recipient co-exist in the recipient, and are both recognized as “self” and not rejected. The terms are further intended to encompass trimeric and multimeric states such as, but not limited to, (a) states in which the recipient may have cells exhibiting both donor and recipient surface histocompatibility antigens, as well as cells from a third or multiple additional donors that are recognized as “self” by the recipient, all co-existing in the recipient; (b) states in which the recipient may have cells from three or multiple donors that are recognized as “self” by the chimeric recipient; and (c) all possible combinations and permutations of the foregoing, without limitation.

As used herein, the term “crude,” when referring to donor bone marrow, is intended to encompass bone marrow that is harvested from a bone and has not necessarily undergone further processing. Preferably, the crude donor bone marrow is unprocessed marrow which includes stromal cells, mesenchymal cells, etc. Therefore, the crude bone marrow can include natural microanatomical components of the bone marrow including pluripotent stem cells, progenitor cells (including early progenitor cells), extracellular matrix, stromal elements, mesenchymal cells, and the like, normally present in bone marrow.

As used herein, “long term tolerance” means a period of time greater than 100 days, preferably greater than 300 days, more preferably greater than 720 days and, most preferably, lifelong survival of the allograft after cessation of the treatments.

As used herein, the term “mixed donor-recipient chimerism,” and like terms, are used to described a state in which tissue or cells from a donor are able to live and function within a recipient host without rejection or the occurrence of GVHD. In a semi-allogeneic transplantation, where the donor and the recipient share at least one major histocompatibility complex (MHC) class I or class II locus, and the chimeric cells exhibit cell surface histocompatibility antigens of both the donor and the recipient (i.e., they are double positive). In a fully allogeneic transplantation, the donor and recipient do not share an MHC locus. In these chimeras, cells from the donor and cells from the recipient co-exist in the recipient, and these are both recognized as “self” and not rejected.

As used herein, “prophylactic” means the protection, in whole or in part, against allograft rejection.

As used herein, the term “suspension,” when referring to bone marrow cells, is intended to encompass cells fluidly flushed from the bone marrow cavity and/or a suspension of cells obtained from processed crude bone marrow.

As used herein, “therapeutic” means the amelioration of allograft rejection itself, and the protection, in whole or in part, against further allograft rejection.

As used herein, a “therapeutically effective amount” of an immunosuppressive agent or an anti-T cell antibody is that amount which is sufficient to deplete T cells.

As used herein, a “therapeutically effective amount” of bone marrow cells is that amount sufficient to induce allograft tolerance and/or the production of mixed donor-recipient chimeric cells in the recipient. The term is intended to encompass an amount by weight of crude bone marrow and/or a number of isolated cells or subpopulation of isolated cells in suspension. Preferably, the therapeutically effective amount of the implanted bone marrow cells is an amount sufficient to maintain long-term recipient tolerance of the allograft without the necessity of readministration of the immunosuppressive agent and the anti-αβ T cell receptor antibodies.

### 4. BRIEF DESCRIPTION OF THE DRAWINGS

FIGS. 1A-1C illustrates the level of chimerism after transplantation of 35×10⁶ or 70×10⁶ donor-derived stem cells, delivered intravenously or directly into the bone (intraosseous). FIG. 1A shows the level of chimerism in the recipient at day 63 for recipients receiving no CsA/αβTCR antibody therapy. FIG. 1B shows the level of chimerism present in recipients receiving combination CsA antibody monotherapy. FIG. 1C shows the level of chimerism present in recipients receiving combination CsA/αβTCR antibody therapy.

FIG. 2 shows the percentage of bone marrow cells in the donor bone and in the host bone at day seven following vascularized bone marrow transplantation in MHC mismatched subjects. Bone marrow cells were stained for CD90, CD90 and CD45, or CD90 and CD3, and the percentage of different cell phenotypes (CD90, CD90/CD3 and CD90/45RA) are presented for host and grafted bone without treatment and under CsA/TCR protocol.

FIG. 3 illustrates donor-specific (for CD4, CD8 and CD45RA) chimerism levels in the recipients of bone allograft transplants at day 7 post-transplant evaluated by flow cytometry.

FIG. 4 shows the kinetics of chimerism from day 7 until day 63 post transplantation of a vascularized bone marrow allograft across and MHC barrier, under combined CsA and αβ TCR antibody protocol.

FIG. 5 shows the histological status of bone marrow allografts at day 7 post-transplant in the isograft (FIG. 5A), CsA/αβ TCR therapy (FIG. 5B), and no-treatment (FIG. 5C) groups. Allografts were vascularized bone marrow transplants across an MHC barrier.

FIG. 6 shows comparison of chimerism for CD4⁺ lymphocytes (upper chart) and CD8⁺ lymphocytes (lower chart) between vascularized bone marrow and hind limb transplant at days 7, 21, 35 and 63 post-transplantation.

FIG. 7 illustrates flow cytometric (FC) evaluation of the peripheral blood αβ TCR⁺ T cells in recipients receiving skin and crude bone marrow semi-allogeneic transplants and treated with combined CsA/αβ TCR mAb immunosuppressive therapy. FC analysis at day 7 post-transplantation demonstrated >90% depletion of the αβ TCR⁺ T cells, with gradual reconstitution to pre-transplant levels at day 63 post-transplantation. Sentinel (untransplanted) animals treated with the combined CsA/αβ TCR mAb served as controls.

FIG. 8 illustrates FC determination of the donor-originated RT-1ⁿ expression on CD4⁺ (A) and CD8⁺ (B) T cell subpopulations isolated from peripheral blood of recipients of skin and crude bone marrow transplantation and treated with combined CsA/αβ TCR mAb immunosuppressive therapy. Examination of two-color stained RT-1^{n-FITC}/CD4^{-PE} and RT-1^{n-FITC}/CD8^{-PE} peripheral lymphocytes revealed 4.1% and 7.4%, respectively, of double positive CD4 and CD8 T cell subpopulations.

FIG. 9 illustrates FC analysis of the CD90⁺ antigen expression on the surface of stem and progenitor cells from donor bone marrow before and after selection using a magnetic MiniMACS separating system. FIG. 9A shows CD90⁺ cells as the small peak, and the remainder of the bone marrow nucleated cells as the larger peak, prior to selective separation of the CD90⁺ cells from a suspension of bone marrow cells. The CD90⁺ cells comprised about 22% of the bone marrow nucleated cells. FIG. 9B shows the purity of separation of the CD90⁺ cells (large peak). Less than 5% were CD90⁻. Over 95% of analyzed cells expressed the CD90 antigen, indicating high efficacy of the selection.

FIG. 10 illustrates intraosseous transplantation of the donor-derived stem and progenitor cells. FIG. 10A illustrates the injection of the stem and progenitor cells directly into the bone marrow cavity of the recipient's left tibia. Following injection, the right hindlimb from the same donor was transplanted to the recipient (FIG. 10B).

FIG. 11 illustrates a hindlimb allograft survival chart indicating significant extension of hindlimb allograft survival (p<0.05) following perioperative injection of 8-12×10⁵ stem and progenitor cells (CD90⁺ cells) directly into the bone marrow cavity of the hindlimb allograft recipients without an immunosuppressive protocol.

FIG. 12 illustrates a two-color flow cytometric analysis at day 14 after hindlimb allograft transplantation, showing transient chimerism in the allograft control treatment (0.6%, A) and a high level (3.4%, B) of double positive RT-1^{l+n}/CD4⁺ chimeric cells in the peripheral blood of limb recipients treated with the intraosseous injection of donor CD90⁺ stem and progenitor cells at the time of transplantation.

FIG. 13 illustrates flow cytometric analysis at the day 35 after hindlimb allograft transplantation, showing high levels of multilineage donor-specific lymphoid chimerism (13B1-13B3) in the peripheral blood mononuclear cells (PMBC) of the recipients receiving direct intraosseous injection of donor stem and progenitor cell. In contrast, intravenous injection of the same number of cells resulted in low-level, transient chimerism (13A1-13A3), indicating that bone creates more permissive conditions for donor stem and progenitor cell engraftment.

FIG. 14 illustrates vascularized skin and bone allografts (VSBA). A schematic representation of the VSBA model combining a superficial epigastric skin flap and a vascularized femoral bone allograft (14A). FIG. 14B shows a Giemsa stained vascularized bone marrow isograft 7 days after transplantation, showing over 99% viability of the bone marrow cells in these transplants. FIG. 14C shows an accepted VSBA transplant allograft across a fully mismatched major MHC barrier (Brown Norway donor, Lewis recipient) at day 63 after cessation of immunosuppressive protocol, showing full acceptance of the vascularized skin allograft. FIG. 14D shows the skin biopsy (hematoxylin and eosin stained) with preserved dermis and epidermis and no histological signs of rejection. FIG. 14E shows the immunohistostained frozen sections of the bone marrow isolated from the donor vascularized bone allograft after transplantation into the recipient, at day 63 after cessation of immunosuppressive protocol. FIG. 14F illustrates flow cytometry analysis of isolated cells. More than 50% of the cells in the donor bone marrow were recipient CD90⁺/RT-1^{L} (related to LEW MHC class I) positive cells, showing the trafficking of recipient cells into the donor bone marrow, and also confirming the viability of the transplanted vascularized bone marrow.

FIG. 15 illustrates a LEW recipient of two genetically unrelated VSBA transplants (i.e., transplantation across a major histocompatibility barrier) at day 35 after transplantation. The vascularized bone transplants are not visible, as they are beneath the transplanted skin flaps shown. On the left is a VSBA transplant from a BN donor, showing full skin acceptance by the LEW recipient of this fully allogeneic transplant. On the right is a VSBA transplant from an ACI donor, showing full skin acceptance by the LEW recipient of this fully allogeneic transplant.

FIGS. 16A and 16B illustrate H&E stained formalin-fixed skin tissues taken from the BN allograft and the ACI allograft, respectively, at day 21 after transplantation, showing preserved dermis and epidermis and no histological signs of rejection.

FIG. 17 illustrates flow cytometry analysis performed on PBMC of the LEW recipients at day 21 after transplantation of VSBA transplants from two fully-mismatched donors, BN and ACI. The dot-plot results of the lymphoid cell subpopulations obtained from quadrangle analytical gates demonstrated the presence of double positive RT-1^{a-FITC}/CD4^{-PE}, RT-1^{a-FITC}/CD8^{-PE} and RT-1^{a-FITC}/CD45RA^{-PE} (ACI/LEW) at a level of 8.02%, 4.36% and 0.82%, respectively, of PBMC (top horizontal row); and also the presence of double positive RT-1^{n-Cy7}/CD4^{-PE}, RT-1^{n-Cy7}/CD8^{-PE} and RT-1^{n-Cy7}/CD45RA^{-PE} (BN/LEW) at a level of 0.9%, 0.3% and 4.1%, respectively (bottom horizontal row), indicating trimerism.

FIG. 18 illustrates flow cytometry analysis performed on PBMC of the LEW recipients at day 35 after transplantation of VSBA transplants from two fully mismatched donors, BN and ACI. The dot plot results demonstrated the presence of double positive RT-1^{a-FITC}/CD4^{-PE}, RT-1^{a-FITC}/CD8^{-PE} and RT-1^{a-FITC}/CD45RA^{-PE} (ACI/LEW) at a level of 7.99%, 4.73% and 0.6%, respectively, of PBMC (top horizontal row); and also the presence of double positive RT-1^{n-Cy7}/CD4^{-PE}, RT-1^{n-Cy7}/CD8^{-PE} and RT-1^{n-Cy7}/CD45RA^{-PE} (BN/LEW) at a level of 0.8%, 0.48% and 3.1%, respectively (bottom horizontal row), indicating trimerism.

### 5. DETAILED DESCRIPTION OF THE INVENTION

### 5.1 Introduction

The present invention provides new methods for achieving long-term survival of even fully mismatched allografts, without chronic immunosuppression and without the requirement for toxic recipient preconditioning, without the need for chronic immunosuppressive therapy, and without the occurrence of GVHD. The methods described and claimed herein are especially useful for human patients requiring transplantation of non-vital organs, including, but not limited to, those needing skin replacement after devastating burn injuries, cancer patients who need customized replacement of large parts of their bodies, immobilized rheumatoid patients requiring replacement of several joints, children born with congenital defects, and the like. Moreover, embodiments of the methods of the invention can be useful for solid organ and gland transplants, and allografts for treatment of inborn errors of metabolism, leukemias, immunodeficiency syndromes, GVHD relapse, and the like, without limitation. Significantly, the present invention allows the transplantation of organs or composite tissue allografts from fully MHC mismatched donors. This will greatly increase the availability of organs or other body parts for transplantation and thus help alleviate the problems associated with severe organ donation shortages.

T cell recognition of foreign major histocompatibility complex (MHC) antigens plays a crucial role in the initiation of allograft rejection. T lymphocytes are classified as αβ or γδ depending on the type of disulfide-linked heterodimeric glycoprotein T cell receptor (TCR) displayed. The T cells responsible for most immune responses, including allograft rejection, are the T cells bearing αβ T cell receptors (αβ TCR⁺ T cells). It was discovered that anti-αβ TCR antibodies, preferably monoclonal (mAb) anti-αβ TCR antibodies, can successfully be employed to specifically eliminate αβ TCR⁺ T cells to create a window of immunological incompetence in the transplant recipient. While not being bound by theory, it is believed that subsequent repopulation of the recipient thymus by αβ TCR⁺ T cells, in the presence of donor alloantigens, results in the induction of hematopoietic mixed donor-recipient chimerism in the transplant recipient and the long-term immunological tolerance demonstrated in these recipients.

Treatment with the anti-αβ TCR mAb alone reduces T cell numbers in a dose-dependent manner, with a significant reduction after one dose of antibody; however T cell depletion does not progress further with continued antibody therapy. While not being bound by theory, it is believed that some T cells may escape initial exposure to the depleting antibody and repopulate rapidly to reject the graft. To address this apparent “T cell escape phenomenon,” an immunosuppressive agent, in addition to the anti-αβ TCR mAb, is employed to prevent the rejection response by reducing allograft-responsive T cell proliferation and enhancing the effectiveness of the depletion protocol.

### 5.2 Methods of Transplantation: Delivery of Bone Marrow into Bone

It is an objective of the present invention to provide a new clinically applicable method of stem cell transplantation which facilitates engraftment and reconstitutes immunocompetence of the recipient without recipient conditioning, or development of GVHD or graft rejection. Aspects of the present invention are based on the discovery that the presence of certain structural components of the bone marrow facilitate efficient engraftment of HSCs. In particular, the present invention combines the use of an immunosuppressant and an anti-αβ T cell receptor antibody, in combination with the administration to the recipient of donor stem cells contained within bone marrow, particularly crude or unprocessed bone marrow, or contained within a bone marrow cavity, or scaffold or substitute, resulting in efficient, long-term engraftment and tolerance.

In one embodiment according to the invention, a method is provided for inducing donor-specific tolerance and/or mixed donor-recipient chimerism in an allograft transplant recipient, comprising the steps of: (a) administering to a recipient of an allograft a therapeutically effective amount of an immunosuppressive agent that depletes T cells; (b) administering to the recipient of the allograft a therapeutically effective amount of anti-αβ T cell receptor antibodies; (c) administering to the recipient a therapeutically effective amount of donor stem cells, wherein said immunosuppressive agent, anti-αβ T cell receptor antibodies and stem cells induces donor-specific tolerance and/or mixed donor-recipient chimerism in said allograft transplant recipient. Preferably, the stem cells are donor hematopoietic stem cells.

In another embodiment of the invention, a method is provided for inducing donor-specific tolerance and/or mixed donor-recipient trimerism in an allograft transplant recipient, comprising the steps of: (a) administering to a recipient of a first allograft and a second allograft a therapeutically effective amount of an immunosuppressive agent that depletes T cells; (b) administering to the recipient of the allograft a therapeutically effective amount of anti-αβ T cell receptor antibodies; (c) implanting a therapeutically effective amount of bone marrow cells from the first allograft donor into the recipient; and (d) implanting a therapeutically effective amount of bone marrow cells from the second allograft donor into the allograft recipient, wherein said immunosuppressive agent, anti-αβ T cell receptor antibody, and bone marrow cells from said first donor and from said second donor induce a state of trimerism in the recipient. The first and second allogeneic allograft donors are preferably independently selected from semi-allogeneic donors; fully allogeneic donors; and combinations thereof, with respect to the recipient. Preferably, the state of trimerism in the recipient comprises a level of mixed donor and recipient cells of about 0.1% to about 15%, preferably about 1% to about 10%, of circulating peripheral blood mononuclear cells.

This embodiment of the invention is particularly applicable to human transplantation. For example, a patient could receive a solid organ transplant, such as a heart, for example, from one unrelated donor and, later, could receive a kidney from a different unrelated donor. This scenario would produce a state of classical trimerism in the recipient, with co-existing recipient cells, and cells from the donor of the heart and from the donor of the kidney, all co-existing without organ rejection in the recipient. As a further permutation, the same patient could receive another kidney from yet another unrelated donor producing a state in which the cells of the recipient and of three independent donors co-exist in the recipient. This would be a state of “multichimerism.”

In another non-limiting example, a patient could receive a solid organ transplant, such as a kidney, from a related donor that shares one or more MHC loci with the recipient, and a heart transplant from an unrelated donor. This scenario would produce a state of trimerism in the recipient, with cells expressing both donor and recipient antigens co-existing with cells from the related donor. A later transplant from a different related donor that shares a different MHC locus with the recipient, or unrelated donor would produce a state of multichimerism.

The methods according to the invention are fully applicable to transplantation of any type of allograft including, but not limited to, composite tissue such as, but not limited to human hand, human finger, human larynx, joints such as knee, hip, limbs, lower extremities, and the like; solid organs and glands such as, but not limited to, heart, lung, kidney, liver, pancreas, thyroid, and the like; glandular cells such as, but not limited to, islet cells and the like; skin; cartilage, such as an ear; hematopoietic tissue; lymphoid tissue; tendons; ligaments; muscles; nerve tissue vascular tissue such as vessels; and the like, without limitation. Additionally, the method, particularly the intraosseous delivery of bone marrow and/or bone marrow-derived cells as described elsewhere herein, is useful for the treatment of blood diseases such as leukemia, or conditions such as genetic immunodeficiencies.

The method of inducing graft tolerance and of promoting chimerism within the recipient, as described herein, is useful for the initial conditioning of a transplant recipient prior to or at the time of the transplant. The method is also useful for the re-establishment of chimerism at any point after transplantation. In particular, treatment of an allograft transplant recipient may include re-administration of the immunosuppressant, anti-T cell receptor antibody and bone marrow (or bone marrow substitute, scaffold, purified bone marrow progenitor or stem or mesenchymal cells, etc.), where the transplant recipient begins to show signs of rejection of the allograft. That is, the methods may be used therapeutically, to reverse the symptoms of, or the progression of, graft rejection, as well as prophylactically, to reduce or eliminate graft rejection and the onset of graft-versus host disease in the recipient.

Cells, such as bone marrow-derived stem cells, progenitor cells or mesenchymal cells, for example hematopoietic stem or progenitor cells, or mesenchymal cells, contained within bone marrow transplanted into the bone of a recipient do not stay compartmentalized within the donor bone marrow, but migrate and/or proliferate to the adjacent recipient bone. Intraosseous delivery thus provides a means of establishing chimerism within the transplant recipient that does not require systemic delivery or recipient conditioning by, for example, myeloablation.

The donor stem cells of the above method may be delivered by any acceptable means that results in systemic delivery. However, delivery of the donor stem cells is best accomplished by intraosseous delivery. The intraosseous delivery of donor hematopoietic stem cells can be performed by direct injection, such as by syringe, into the bone marrow of the recipient of a therapeutically effective amount of donor hematopoietic stem cells. This can be accomplished by the use of minimally invasive techniques involving drilling a small hole into the recipient's bone, flushing out a small volume of recipient bone marrow to create the “space” for the recipient bone marrow, and injecting the donor bone marrow into the small cavity created. Alternatively, a pumping system may be used for the intraosseous transfer of hematopoietic stem cells. Any pumping system that is medically and pharmaceutically acceptable, and which can transfer cell from donor to recipient, may be used. For example, a pressurized pumping system may be used wherein recipient bone marrow is pumped out and donor cells and/or marrow is pumped in to the recipient bone. The hematopoietic stem cells isolated from the donor may be delivered in crude form, i.e. they may comprise other elements of the donor bone marrow. Alternatively, the hematopoietic stem cells may be purified and enriched, such as by cell sorting techniques that are well known in the art, prior to intraosseous delivery. Transplantation of vascularized bone containing bone marrow may also be used to effect transplantation of bone marrow, and stem cells contained therein, for example, mesenchymal cells, B cells, chimeric cells derived from the recipient, etc.

Markedly enhanced allograft survival and mixed chimerism can be achieved by intraosseous implantation of the donor bone marrow cells employing any delivery system including, but not limited to, an implantable, vascularizable bone from the allograft donor including the donor bone marrow; implantable crude donor bone marrow; an implantable donor bone marrow cell suspension; an implantable isolated subpopulation of nucleated donor bone marrow cells; and combinations thereof.

In a preferred embodiment of the invention, a method is provided for donor bone marrow transplantation directly into the medullary cavity of the transplant recipient bone. This method allows avoidance of recipient conditioning for creation of immunological space, and preservation of the microenvironment for the bone marrow cells during transplantation. The method provides for direct engraftment of donor-derived bone marrow stem and progenitor cells and, as a result, the establishment of donor-specific hematopoietic chimerism. That is, the method provides preservation of the donor bone marrow microenvironment for creation of a chimeric state in the recipient without the need for recipient preconditioning (such as whole body irradiation or the like) or for vascularized bone marrow transplantation, such as by composite allograft transplants, vascularized bone implantation, or the like.

The direct transplantation of the donor bone marrow in the crude form into the medullary cavity of the long bones of transplant recipient allows not only for the engraftment of the bone marrow cells, but also provides a natural matrix as an ideal microenvironment for cell engraftment for subsequent cell repopulation and trafficking. As described in the examples below, the engraftment of the donor specific bone marrow cells and their trafficking into the periphery was confirmed by flow cytometry, revealing 4.1% and 7.4% of donor specific RT-1ⁿ-FITC/CD4⁻PE and RT-1ⁿ-FITC/CD8⁻PE chimeric cells, respectively, in the peripheral blood of recipients at day 63 after cessation of the immunosuppressive treatment protocol. The induction of this mixed, donor specific chimerism resulted a the significant extension of the skin allograft survival in recipients of the donor crude bone marrow. This method of crude bone marrow transplantation is simple, minimally invasive, does not require recipient preconditioning and can be easily implemented into clinical practice in humans.

The step of implanting donor bone marrow cells into the recipient by direct intraosseous transfer of donor crude bone marrow can comprise the substeps of obtaining crude bone marrow from a bone marrow cavity of the allograft donor, and implanting the donor crude bone marrow into the allograft recipient. Any suitable method can be employed to obtain the crude bone marrow such as, but not limited to, scooping the marrow from the donor bone and depositing the scooped marrow directly into the bone marrow cavity of a recipient bone. Optionally, it may be desirable to create a space in the recipient bone marrow cavity prior to receiving the donor bone marrow. As a non-limiting example, it may be desirable to remove a roughly equivalent amount of recipient marrow to that of the donor marrow to be received. However, neither creation of the space nor the amount of recipient bone marrow removed are critical to the present invention.

In another embodiment of obtaining crude donor bone marrow and implanting the same into a recipient bone marrow cavity, a pumping device may be employed. That is, an amount of recipient marrow can be withdrawn through one cannula, and an amount of donor marrow can be implanted through another cannula. These steps can be repeated as often as necessary to complete the delivery of a desired amount of the crude donor bone marrow. The pumping device can be any suitable device, including a two-way syringe mechanism, without limitation. In embodiments in which a two-way syringe is used, it may be a syringe in which the amount withdrawn or delivered, and/or the pressure for withdrawal or delivery, is regulatable.

The amount of donor crude bone marrow to be delivered to the recipient will depend on numerous factors such as, but not limited to, the type and condition of the recipient, the route of administration, and the like as described above, and can be tailored to each recipient according to normal medical practice. A therapeutically effective amount of donor crude bone marrow implanted into the recipient is that sufficient to induce allograft tolerance and/or the production of mixed donor-recipient chimeric cells in the recipient.

In another embodiment of the invention, the step of implantation of the donor bone marrow cells comprises the substeps of obtaining a bone marrow cell suspension from a bone of the allograft donor, and implanting the bone marrow cell suspension into the recipient. The suspension of bone marrow cells can be prepared by fluidly flushing the bone marrow cells from the bone marrow cavity is intended to encompass cells fluidly flushed from the bone marrow cavity and/or a suspension of cells obtained by processing crude bone marrow. The former flushing method is preferred. In a non-limiting example, freshly isolated donor bone such as, but not limited to, the humerus, femur, tibia, and the like, are isolated and two contralateral ends of the bones can be cut and the residual bone marrow cells flushed out from the bone marrow cavity using a syringe-based pump system. After lysis of red blood cells, nucleated marrow cells are then washed and counted to obtain a final concentration of cells in a physiological medium, such as Medium 199 with gentamycin; phosphate buffered saline, or the like.

The suspension of bone marrow cells is then employed for implantation into the allograft recipient by direct intraosseous transplantation into a recipient bone marrow cavity or by intravenous injection into the recipient. Alternatively, as described further below, implantation into the allograft recipient can be by direct intraosseous transplantation into an implanted vascularized donor bone or donor bone scaffold or commercially-available bone allograft.

Donor stem cells, either purified or in the form of a bone marrow suspension, may first be seeded in a commercially available bone scaffold and propagated for repeated delivery to a recipient. In this embodiment, depending on whether the bone scaffold is implantable, the isolated donor hematopoietic stem cells may further be seeded in the bone scaffold and subsequently transplanted together with the bone scaffold into the recipient by intraosseous delivery. Any medically and/or pharmaceutically-acceptable bone scaffold may be used in the present invention. Commercially available bone scaffolds are well known to persons of skill in the art and may comprise biologically active materials (such as live or vascularized donor bone and bone marrow) and biologically inactive materials, such as synthetic or engineered biomaterials, including demineralized bone matrix, fibers, gels and foams. Without limitation, examples of such scaffolds include hydroxyapatite ceramics such as ApaPore®, porous zirconia (ZrO₂) scaffolds, bioresorbable polyurethane scaffolds, resorbable polylactide scaffolds impregnated with bone marrow, etc.

In the embodiments of the invention, the stem cell or bone marrow donor can be a mammal of a first species and the recipient can be a mammal of a second species. In another embodiment, the donor and the recipient can be of the same species. In other embodiments, donor and recipient are mammals, birds, reptiles, amphibians or marsupials. In a specific embodiment, said mammals are domestic mammals. In a more specific embodiment, said domestic mammal is a canine or a feline. In another more specific embodiment, said domestic mammal is an equine, bovine, porcine species. In yet further embodiments, the recipient is a primate. In a preferred embodiment, the recipient is a human.

In other embodiments, donor and recipient may be genetically unrelated individuals, or may be from the same immediate family. In other embodiments, donor and recipient may share 1 or more HLA markers, or other immune-determining markers, or may share none.

Encompassed within the invention is the intraosseous transfer of bone marrow, or a crude bone marrow cell preparation, where the transplanted material is the bone marrow or bone marrow cell preparation per se, and where the transplanted bone marrow or bone marrow cell preparation is an adjunct to the transplantation of an allograft, for example, a composite tissue allograft.

In another embodiment, the method may also include the transplantation of a “monitor” allograft used to assess the state of the primary allograft post-transplantation. While the monitor allograft need not be transplanted at the same time as the primary allograft, preferably they are transplanted at the same time (i.e., on the same day). Preferably, the monitor allograft is a patch of donor skin, preferably non-vascularized, that is transplanted to the allograft recipient. Also preferred is the transplantation of artificially or commercially-available skin or skin substitute seeded with donor keratinocytes. Because skin is most easily rejected, the “monitor” allograft will most likely be affected first should graft tolerance diminish and rejection and/or graft versus host disease initiate. Such signs include, for example, edema, swelling, necrosis, particularly at the margin of the monitor graft, etc. Because the monitor allograft is affected before the primary allograft, rejection of the primary allograft may be averted by re-establishment of immune tolerance and/or recipient chimerism, as described herein. Thus, the invention provides for a method of monitoring the status of a primary allograft in a recipient that has received the primary and a monitor allograft, comprising examining the monitor allograft for a plurality of days after transplantation of said primary allograft and said monitor allograft; determining whether the monitor allograft exhibits one or more signs of rejection or graft versus host disease; and, if so, administering an immunosuppressant, an anti-T cell receptor antibody and a plurality of bone marrow cells derived from the donor of said allograft, in an amount sufficient to reduce said signs of rejection or graft versus host disease. In a specific embodiment, said plurality of bone marrow cells are contained within unprocessed bone marrow or a crude bone marrow suspension.

### 5.3 T Cell Depletion

5.3.1 Immunosuppressants and Anti-αβ T Cell Receptor Antibodies

The method of the present invention comprises the depletion of αβ T cells by the administration of at least one immunosuppressant and at least one anti-αβ T cell receptor antibody.

An immunosuppressive agent, as used herein, is an agent such as a chemical agent or a drug that, when administered at an appropriate dosage over an appropriate time period, results in the depletion of T cells, preferably mature T cells. The immunosuppressive agent is preferably an inhibitor of the calcineurin pathway of T cell activation such as, but not limited to, cyclosporine A (CsA), FK-506, and the like; or other inhibitors of IL-2 production such as, but not limited to, rapamycin and the like, and combinations of the foregoing. More preferably, the immunosuppressive agent is CsA. The methods of the invention also encompass the use of a plurality of immunosuppressants. In one embodiment, said plurality of immunosuppressants is the combination of CsA and an IL-2 inhibitor.

Any αβ T cell receptor antibody that can effect depletion of recipient αβ T cells may be used in the methods of the invention. Such antibodies may be monoclonal or polyclonal. In particular, non-monoclonal anti-αβ T cell receptor antibodies with suitable specificity and an efficacy similar to monoclonal αβ T cell receptor antibodies, or whose epitope overlaps that of the monoclonal antibody, are suitable. However, the antibodies are preferably monoclonal (mAb) αβ T cell receptor antibodies, and such monoclonal antibodies are generally commercially available. Preferably, the αβ T cell receptor antibody is from the same species as the recipient; however, an antibody from any species, particularly any mammalian species, may be used in a recipient of any species, particularly a mammalian species. Preferably, a human αβ T cell receptor antibody is used for the depletion of human αβ T cells. In a preferred embodiment in the rat model, the antibody is Clone R73 (Medimmune). In another embodiment, the antibody is a mouse anti-rat antibody. In a specific example, the mouse anti-rat antibody is antibody R73. In another preferred embodiment, the antibody against the human T cell receptor is a murine antibody. In a specific embodiment, the anti-T cell receptor antibody is the mouse anti-human antibody designated MEDI-500 (T10B9.1A-31; BD Pharmingen). In other embodiments, the anti-T cell receptor antibody is a recombinant antibody. In a specific example, the recombinant antibody is produced by a plant. In an even more specific example, the plant-produced antibody is produced by a corn plant.

Where monoclonal antibodies are used, the method of the invention encompasses the use of a plurality of monoclonal antibodies. In one embodiment, for example, the method encompasses the use of a plurality of monoclonal antibodies to a human αβ T cell receptor, each of which recognizes a unique epitope. The invention also encompasses the use of a monoclonal antibody with a polyclonal antibody preparation, or, alternatively, the use of a plurality of polyclonal antibody preparations.

αβ T cell receptor antibodies useful in the present invention may also be produced by known methods without undue experimentation. It is also known that hybridomas producing monoclonal antibodies may be subject to genetic mutation or other changes while still retaining the ability to produce monoclonal antibody of the same desired specificity. The embodiments of the invention methods therefore encompass mutants, other derivatives and descendants of the hybridomas producing anti-αβ TCR mAbs. It is also known that a monoclonal antibody can be subjected to the techniques of recombinant DNA technology to produce other derivative antibodies, humanized or chimeric molecules or antibody fragments that retain the specificity of the original monoclonal antibody. Such techniques may involve combining DNA encoding the immunoglobulin variable region, or the complementarity determining regions (CDRs) of the monoclonal antibody with DNA coding the constant regions, or the constant regions plus framework regions, of a different immunoglobulin, for example, to convert a mouse-derived monoclonal antibody into one having largely human immunoglobulin characteristics. (See, for example, EP 184187A and GB 2188638A.) The embodiments of the invention also encompass humanized monoclonal antibodies to the αβ TCR epitopes.

The immunosuppressive agent(s) and/or the antibodies useful in embodiments of the invention can be a pharmaceutically acceptable analogue or prodrug thereof, or a pharmaceutically acceptable salt of the immunosuppressive agent(s) or antibodies disclosed herein, which are effective in inducing long-term, donor specific tolerance to allografts. By prodrug is meant one that can be converted to an active agent in or around the site to be treated.

5.3.2 Administration of Immunosuppressants and αβ-T Cell Receptor Antibodies

In embodiments of the present invention, administration of the therapeutically effective amount of a combination of anti-αβ T cell receptor antibodies and the immunosuppressive agent capable of depleting T cells, preferably mature T cells, can be given prophylactically or therapeutically. The antibodies and immunosuppressive drugs, as used herein, include all biochemical equivalents thereof (i.e., salts, precursors, the basic form, and the like). Preferably, the immunosuppressive agent and the anti-αβ T cell receptor antibodies are administered according to a protocol such as those disclosed and claimed in our co-owned, copending U.S. patent application Ser. No. 10/427,013, filed Apr. 30, 2003, entitled, “Induction and Maintenance of Tolerance to Composite Tissue Allografts,” the entire disclosure of which is hereby incorporated by reference.

The immunosuppressive agent and the anti-αβ T cell receptor antibodies are preferably administered in an amount, at a frequency, and for a duration of time sufficient to induce donor-specific tolerance and/or mixed donor-recipient chimerism in the allograft recipient.

The combination of immunosuppressant and anti-αβ T cell receptor antibody is administered in an amount effective to deplete T-cells; that is, to eliminate about 50% to about 99.9%, preferably about 75% to about 95%, more preferably about 80% to at least about 90% of the αβ TCR⁺ cells during the short course of therapy. The embodiments of the method of the invention provide significant depletion of the recipient T-cell population at the end of the immunodepleting therapy, as well as allow repopulation of the recipient T cell repertoire once the treatment protocol is withdrawn. In another embodiment, the immunosuppressive T cell deleting agent and the anti-αβ TCR⁺ T cell receptor antibodies are administered as a pharmaceutical composition that comprises both the immunosuppressive agent and the antibodies.

In a preferred embodiment, the immunosuppressive agent and the anti-αβ T cell receptor antibodies are administered as a short course of therapy that can be initiated prior to transplantation, alternatively at transplantation, alternatively about one to about three days after transplantation and, preferably, continues for a short time period after transplantation. For example, administration of the immunosuppressant and the anti T cell antibodies may continue for 1, 2, 3, 4, 5, 7, 10 or 20 days after transplantation. In an embodiment of the invention that is particularly useful for semi-allogeneic transplantation, the immunosuppressive agent and the anti-αβ T cell receptor antibodies can be initially administered at about the time of transplantation to about 24 hours prior to transplantation, preferably about 12 hours to about 24 hours prior to transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation.

In another embodiment of the invention that is particularly useful for fully-allogeneic transplantation, the immunosuppressive agent and the anti-αβ T cell receptor antibodies are initially administered during a period of time from about one hour prior to transplantation to at the time of transplantation. Administration of the immunosuppressive agent and the anti-αβ T cell receptor antibodies are then administered daily for about 100 days, about 50 days, about 35 days, about 21 days, about 14 days, preferably about 7 days or, especially, for about 5 days after transplantation. For fully allogeneic transplantation, it is more preferable initially to administer the immunosuppressive agent and the anti-αβ T cell receptor antibodies at about the time of transplantation, in order to avoid the occurrence of GVHD in these recipients.

In another embodiment, the immunosuppressive agent and the anti-αβ T cell receptor antibodies are first administered from one to three days after transplantation, and daily administration continues for a period of time of about 100 days, about 50 days, about 35 days, about 21 days, about 14 days, preferably for about 7 days or, especially, for about 5 days after transplantation.

In alternative embodiments, the immunosuppressive agent and the anti-αβ T cell receptor antibodies can be administered independently on a daily and/or non-daily basis during the treatment period of time, depending on the type of transplant, the type of donor, the condition of the recipient, and other factors, according to the judgement of the practitioner as a routine practice, without departing from the scope of the invention. In another embodiment, the immunosuppressive T cell deleting agent and the anti-αβ TCR⁺ T cell receptor antibodies are administered as a pharmaceutical composition that comprises both the agent and the antibodies.

Treatment will depend, in part, upon the particular therapeutic composition used, the amount of the therapeutic composition administered, the route of administration, and the cause and extent, if any, of the disease.

The antibodies and immunosuppressive agent(s) described herein, as well as their biological equivalents or pharmaceutically acceptable salts can be independently or in combination administered by any suitable route. The manner in which the agent is administered is dependent, in part, upon whether the treatment is prophylactic or therapeutic. Although more than one route can be used to administer a particular therapeutic composition, a particular route can provide a more immediate and more effective reaction than another route. Accordingly, the described routes of administration are merely exemplary and are in no way limiting. Suitable routes of administration can include, but are not limited to, oral, topical, subcutaneous and parenteral administration. Examples of parenteral administration include, but are not limited to, intravenous, intraarterial, intramuscular, intraperitoneal, and the like. Preferably, the immunosuppressant and anti-αβ T cell receptor antibody are administered intravenously or subcutaneously.

The dose of immunosuppressive agent, anti-T cell receptor antibodies, and/or donor bone marrow cells administered to a subject, particularly an animal, particularly a mammal, particularly a human, in accordance with embodiments of the invention, should be sufficient to effect the desired response in the animal over a reasonable time frame. It is known that the dosage of therapeutic agents depends upon a variety of factors, including the strength of the particular therapeutic composition employed, the age, species, condition or disease state, and the body weight of the subject, animal, mammal or human. Moreover, the dose and dosage regimen will depend mainly on whether the compositions are being administered for therapeutic or prophylactic purposes, separately or as a mixture, the type of biological damage to the host, the type of host, the history of the host, and the type of immunosuppressive agents or biological active agent. The size of the dose will be determined by the route, timing and frequency of administration as well as the existence, nature and extent of any adverse side effects that might accompany the administration of a particular therapeutic composition and the desired physiological effect. It is also known that various conditions or disease states, in particular, chronic conditions or disease states, may require prolonged treatment involving multiple administrations. Therefore, the amount of the agent and/or antibodies must be effective to achieve an enhanced therapeutic index.

It is noted that humans are generally treated longer than mice and rats with a length proportional to the length of the disease process and drug effectiveness. The therapeutic purpose is achieved when the treated hosts exhibit improvement against disease or infection, including but not limited to improved survival rate of the graft and/or the host, more rapid recovery, or improvement in or elimination of symptoms. If multiple doses are employed, as preferred, the frequency of administration will depend, for example on the type of host and type of disease. The practitioner can ascertain upon routine experimentation which route of administration and frequency of administration are most effective in any particular case. Suitable doses and dosage regimens can be determined by conventionally known range-finding techniques. Generally, treatment is initiated with an amount expected to be in excess of the minimum required to produce the desired depletion of T cells or the desired level of chimerism. Thereafter, the dosage is increased by small increments until the optimum effect under the circumstances is reached.

The dose and dosage regimen will depend mainly on whether the compositions are being administered for therapeutic or prophylactic purposes, separately or as a mixture, the type of biological damage and host, the history of the host, and the type of immunosuppressive agent or biologically active agent. The amount must be effective to achieve an enhanced therapeutic index. It is noted that humans are generally treated longer than rats with a length proportional to the drug effectiveness. The doses may be single doses or multiple doses over a period of several days. Therapeutic purposes are achieved as defined herein when the treated hosts exhibit allograft tolerance, including but not limited to improved allograft survival rate, more rapid recovery, or improvement or elimination of transplantation-associated symptoms. If multiple doses are employed, as preferred, the frequency of administration will depend, for example, on the type of host and type of allograft, dosage amounts, and the like.

In specific embodiments, therefore, the immunosuppressant, anti-T cell antibody, or combination thereof is administered in a dosage that reduces the number of T cells in a recipient to less than 50%, less than 40%, less than 30%, less than 20%, less than 10%, less than 5%, less than 2% or less than 1% of the number of T cells in said recipient prior to administering said immunosuppressant, anti-T cell antibody, or combination thereof.

In specific embodiments, the invention provides for the administration of a sufficient number of donor-derived bone marrow cells (in any of the forms disclosed elsewhere herein) such that the recipient exhibits at least 2% chimerism; at least 5% chimerism; at least 10% chimerism; at least 15% chimerism; at least 20% chimerism; at least 25% chimerism; at least 30% chimerism; at least 40% chimerism; at least 50% chimerism; between 2% and 60% chimerism; between 5% and 50% chimerism; between 10% and 40% chimerism; between 20% and 40% chimerism, etc.

### 5.4 Isolation of Bone Marrow-Derived Cells

Isolated bone marrow cell subpopulations, such as pluripotent stem cells or progenitor cells can be obtained by further processing the foregoing bone marrow suspensions. Isolation methods for cell subpopulations are well known. For example, stem cells and progenitor cells can be separated on the basis of their differential staining with rhodamine; stem cells do not stain with rhodamine, whereas progenitor cells do stain with rhodamine. Exemplary suitable methods for isolation of cell subpopulations include, but are not limited to, separation of subpopulations by a fluorescence activated cell sorter, by positive and/or negative selection by magnetic beads coated with an appropriate antibody, and the like, without limitation. Assessment of the purity of the desired cell populations can be accomplished by flow cytometry, fluorescence-activated cell sorting; antibody-mediated capture; receptor-mediated capture; or other known methods. See Example 13 for a specific example of CD90⁺ cell isolation, which may be extrapolated to the purification of cells with other surface markers of interest.

After isolation of the bone marrow subpopulation, a desired number of cells in suspension can be implanted into the recipient by direct intraosseous injection, as described above, by intravenous routes, or the like, as described for bone marrow suspensions above. Alternately, the cells of the subpopulation may be seeded onto whole bone marrow, or an artificial bone scaffold or bone substitute, as described above.

Preferably, the subpopulation used is bone marrow-derived stem cells. More preferably, these stem cells are hematopoietic stem cells. The Examples, below, exemplify use of rat CD90 cells. However, any equivalent bone marrow-derived stem or progenitor cell population or subpopulation may be used as appropriate for the particular donor and recipient species. For example, any human bone marrow equivalents of human stem cells and progenitor cells such as, but not limited to, CD34⁺, CD19⁺ cells, or the like, can be employed in the human recipient equivalent of the rat model. In other embodiments, primate bone marrow-derived stem or progenitor cells are transplanted into the bone marrow of a primate recipient; canine bone marrow-derived stem or progenitor cells are transplanted into the bone marrow of a canine recipient; feline bone marrow-derived stem or progenitor cells are transplanted into the bone marrow of a feline recipient; etc.

### 5.5 Establishment and Maintenance of Chimerism

In previous studies using a rat hindlimb (CTA) allograft transplantation model (see U.S. patent application publication Nos. 2004/0005315, published Jan. 8, 2004; and 2004/0028677, published Feb. 12, 2004), a remarkably stable hematopoietic donor-recipient chimerism was observed in the recipients as evidenced by a peripheral blood chimeric cell level of about 20% to about 30% of circulating mononuclear cells, and greater than about 60% chimeric cells in lymphoid tissue. Without being bound by theory, this finding suggests that the presence of certain tissue compartments in the composite tissue allografts may facilitate efficient engraftment of the donor hematopoietic cells and contribute to the observed long-term tolerance (over 750 days) across a major histocompatibility complex (MHC) barrier in a multitissue allograft. It is known that bone marrow stromal cells play a critical role in the formation of the hematopoietic microenvironment and support hematopoietic stem cell differentiation through the inter-cellular contact and secretion of various cytokines and growth factors. The hematopoietic microenvironment that is created by transplantation of marrow stromal cells, strains or crude bone marrow allows for the ectopic development of a hematopoietic tissue at the site of transplantation. Again, without being bound by theory, it is possible that microanatomic environment and stromal components in the bone component of the allograft can provide essential support elements that allow for the successful mismatched transplant without recipient preconditioning and contribute to the ability to induce a tolerant state allowing for stable chimerism.

It has unexpectedly been discovered that direct intraosseous implantation of donor derived stem and progenitor bone marrow cells (for example, in the rat model, CD90⁺ cells) into allograft recipients of a CTA from the same donor, in the absence of immunosuppressive therapy and preconditioning, significantly prolonged the survival of the CTA allografts as compared to recipients receiving the allograft only. This survival extension correlated with a transient chimerism of 3.4% of CD4⁺ T cells of donor origin in the peripheral blood of the recipients. Therefore, implantation of donor stem and progenitor bone marrow cells can produce mixed donor-recipient chimerism, even when the recipient is not given an immunosuppressive regimen. This chimerism is associated with increased graft tolerance by the recipient and increased graft survival.

Chimerism may be induced by the transplantation by any acceptable means. However, intraosseous implantation, for example, intraosseous injection, of donor derived stem and progenitor bone marrow cells and intravenous injection of the same number of cells provides superior long-term chimeric stability as compared to other routes of administration, e.g., intravenous administration. Without being bound by theory, maintenance of chimerism depends upon engraftment of donor cells within the microanatomic environment and stromal compartment of the bone marrow of the recipient, followed by migration of the donor-derived bone marrow cells into the lymphoid organs of the recipient for further repopulation and chimeric maintenance.

Although in a rat hindlimb model, about 15% to about 20% chimeric cells were sufficient to achieve indefinite allograft tolerance, other protocols in other animals, including human recipients, may achieve a different peripheral blood level of chimeric cells that is sufficient to achieve indefinite allograft tolerance. Thus, an optimum level of chimerism for maintaining long-term allograft tolerance can vary and can be about 5% to about 50% of circulating PBMC, preferably about 10% to about 40%, more preferably about 15% to about 30%, most preferably about 20% to about 30% of circulating PBMC, without limitation, depending on the individual modality employed. The level of chimerism in lymphoid organs can be as high as about 60% or more and as low as about 25% or less.

Thus, in another embodiment of the invention, a method is provided for maintaining a particular level of mixed donor-recipient chimerism in an allograft transplant recipient, with the goal of prolonging allograft tolerance. In particular, the invention provides a method of maintaining a particular level of chimerism in an allograft recipient, comprising determining an acceptable percentage range of chimerism, wherein the percentage range of chimerism is defined by the upper and lower percentage of donor-derived cells in said allograft; determining, on a plurality of days after transplant of the allograft, the percentage chimerism in said recipient; and, if said percentage chimerism is outside said percentage range of chimerism, administering to said allograft recipient sufficient additional donor-derived cells to raise said percentage chimerism to within said percentage range of chimerism. By the method, an optimum level of chimerism for maintaining long-term allograft tolerance can be determined by measuring an optimal level of chimeric cells in the recipient not undergoing rejection of the allograft. When an optimal level is achieved, the chimeric cells can be harvested from the recipient and stored or, optionally, propagated in cell culture prior to storage. If the recipient later shows signs of allograft rejection, or if the level of chimeric cells is decreasing, or reaches or falls below a minimum level considered sufficient to maintain allograft tolerance, the harvested stored cells are then available to reconstitute the recipient chimeric cells to maintain the tolerant state.

Optionally, the method can include readministering an effective amount of the immunosuppressive agent and/or the anti-αβ T cell receptor antibodies in addition to the chimeric cells. The harvested stored chimeric cells can be administered to the recipient by any method described above including, but not limited to, direct intraosseous injection into a recipient bone marrow cavity; direct intraosseous injection into a bone marrow cavity of an implanted donor bone allograft; intravenous injection into the recipient; injection directly into a target organ; intraperitoneal delivery; and the like, and combinations of the foregoing.

The harvested chimeric cells can be obtained from any recipient tissue or lymphatic organ, but are preferably obtained from the peripheral blood. For example, chimeric cells can be obtained by blood donation from allograft recipients on a regular basis such as, but not limited to, monthly, bimonthly, semi-annually, annually, and the like, for any period of time during which optimal levels are maintained. Chimeric cells may also be harvested after the recipient has been chemically or biologically stimulated to produce more of the chimeric cells.

Peripheral blood mononuclear cells can be separated from the peripheral blood by methods that are well known in the art, and employed for later reconstitution of the recipient. Alternatively, the chimeric cell subpopulations can be separated from the peripheral blood/peripheral blood mononuclear cells by any suitable cell separation method, such as those described above, or the like. Alternatively, the whole peripheral blood can be frozen and stored for later infusion into the recipient.

The harvested PBMC cells and/or chimeric cell subpopulations can be directly stored at −196° C. in liquid nitrogen, or by similar known means. Preferably, the cells are stored in a medium suitable for cryogenic preservation. Alternatively, the harvested cells can undergo expansion in culture prior to storage, using appropriate culture media, feeder cell layers, and the like, that will allow proliferation of the cells. For example, a suitable method for culturing harvested rat cells employs a MyeloCult medium (StemCell Technologies, Vancouver, BC) and rat cell feeder layers in tissue culture dishes. The harvested chimeric cells can be divided and a portion of the harvested cells used to reconstitute the recipient, with the remaining portion stored for later use.

### 5.6 Pharmaceutical Compositions

Compositions for use in the method of the invention preferably comprise a pharmaceutically acceptable carrier and an amount of the therapeutic composition sufficient to induce tolerance prophylactically or therapeutically. The carrier can be any of those conventionally used and is limited only by chemical-physical considerations, such as solubility and lack of reactivity with the compound, and by the route of administration. It will be appreciated by one of ordinary skill in the art that, in addition to the following described pharmaceutical compositions, the therapeutic composition can be formulated as polymeric compositions, inclusion complexes, such as cyclodextrin inclusion complexes, liposomes, microspheres, microcapsules and the like.

The therapeutic composition can be formulated as a pharmaceutically acceptable acid addition salt. Examples of pharmaceutically acceptable acid addition salts for use in the pharmaceutical composition include those derived from mineral acids such as, but not limited to, hydrochloric, hydrobromic, phosphoric, metaphosphoric, nitric and sulfuric acids, and the like, and organic acids such as, but not limited to, tartaric, acetic, citric, malic, lactic, fumaric, benzoic, glycolic, gluconic, succinic, and arylsulphonic, for example p-toluenesulphonic, acids, and the like.

The pharmaceutically acceptable excipients described herein, for example, vehicles, adjuvants, carriers or diluents, are well-known to those who are skilled in the art and are readily available to the public. It is preferred that the pharmaceutically acceptable carrier be one which is chemically inert to the therapeutic composition and one which has no detrimental side effects or toxicity under the conditions of use.

The choice of excipient will be determined in part by the particular therapeutic composition, as well as by the particular method used to administer the composition. Accordingly, there are a wide variety of suitable formulations of the pharmaceutical composition of the present invention. The formulations described herein are merely exemplary and are in no way limiting.

Injectable formulations are among those that are preferred in accordance with the present inventive method. The requirements for effective pharmaceutically carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well-known to those of skill in the art. Such formulations are suitable in the context of the present invention for application to the skin in a form such as, but not limited to, patches, solutions, ointments, and the like.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compositions can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethyl-cellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof. The parenteral formulations will typically contain from about 0.5 to about 25% by weight of the active ingredient in solution. Preservatives and buffers may be used.

In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The present inventive method also can involve the co-administration of other pharmaceutically active compounds. By “co-administration” is meant administration before, concurrently with, e.g., in combination with anti-cancer composition in the same formulation or in separate formulations, or after administration of a therapeutic composition as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered.

### 7. EXAMPLES

To illustrate embodiments of the method of the invention, the following examples employ several allograft transplantation models, immunosuppressive protocols, and delivery systems for donor bone marrow and/or bone marrow cells, including isolated donor stem and progenitor cell populations The examples described herein are not intended to be limiting, as one skilled in the art would recognize from the teachings hereinabove and the following examples that, for example, other immunosuppressive agents, other anti-αβ T cell receptor antibodies, other dosage and treatment schedules, other methods of bone marrow delivery, other isolation methods for donor stem and progenitor cell recovery, other sources of donor stem and progenitor cells, other animal and/or humans, and the like, all without limitation, can be employed, without departing from the scope of the invention as claimed.

### Example 1

Transplantation of donor-derived stem cells can improve organ allograft survival in animal models. This study was designed to investigate the effect of different routes of stem cell transplantation (SCT) on donor specific tolerance induction across MHC barrier under short-term CsA monotherapy and αβ TCR/CsA treatment protocols.

Methods: Forty-eight SCT were performed between BN (RT1ⁿ) donors and Lewis (RT1^{l}) recipients. Intraosseous and intravenous SCT was studied in 6 groups of 8 animals each receiving 35×10⁶ (n=4) and 70×10⁶ (n=4) SCT. Groups I and II served as controls and received SCT but no treatment. Groups III and IV received CsA monotherapy for 7-days, Groups V and VI were under αβ TCR/CsA protocol for 7-day. Flow cytometry analysis was used for evaluation of immunodepletion of T-lymphocytes and multilineage donor specific chimerism for MHC class-I (RT1ⁿ) for RT1ⁿ/CD4, RT1ⁿ/CD8 and RT1ⁿ/CD45RA antigens in peripheral blood of recipients at post-transplant days 7, 21, 35 and 63.

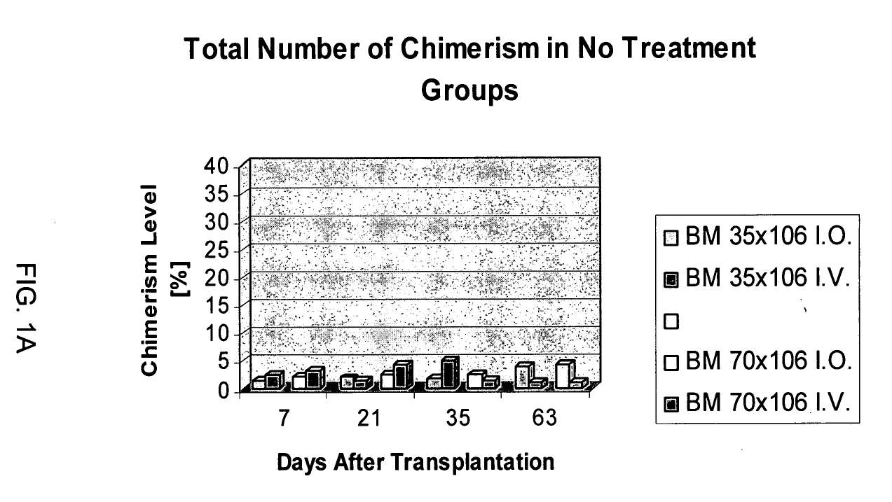

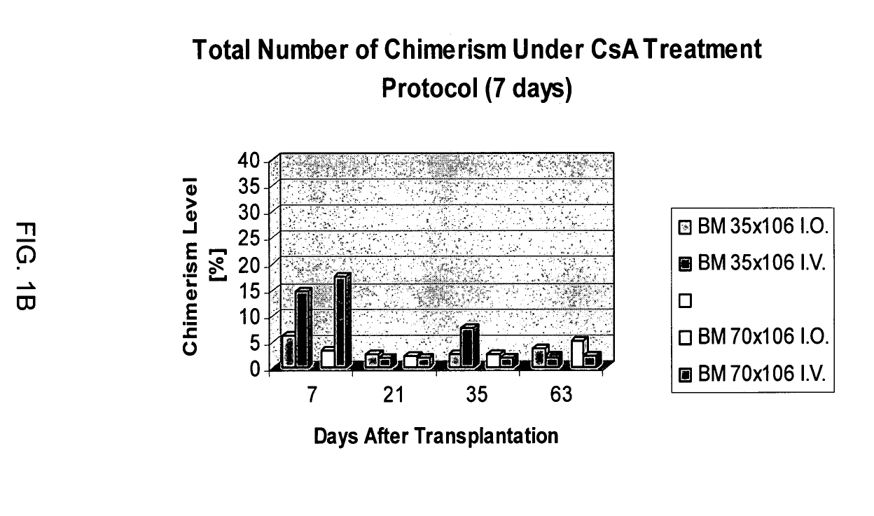

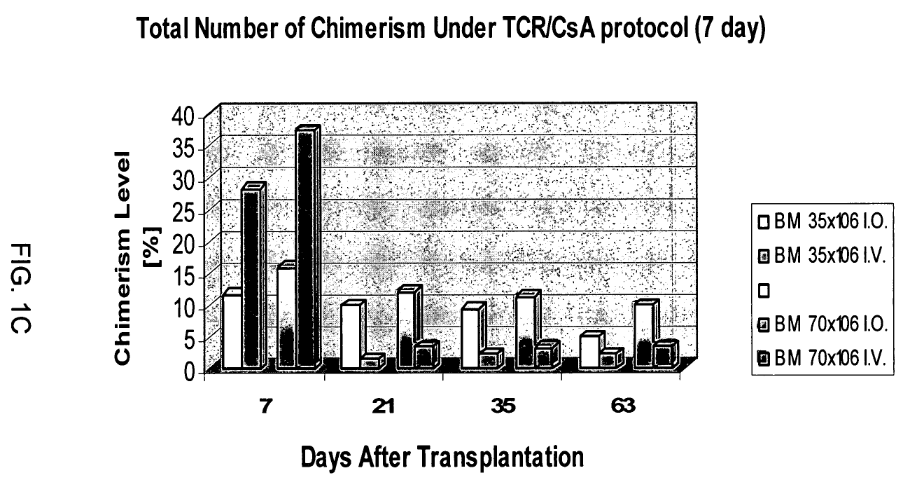

Results: All animals survived without sign of graft versus host disease. At day 63 non-treatment groups receiving 70×10⁶ SCT developed 4.3% of multilineage donor-specific chimerism following intraosseous transplantation and 1.0% following intravenous transplantation. (FIG. 1A)

At day 7 under CsA monotherapy and 70×10⁶ SCT, chimerism level for 1.5% RT1ⁿ/CD4 was induced in intraosseous and 10.5% in intravenous transplanted groups. At day 63 following intraosseous transplantation chimerism for RT1ⁿ/CD4 was stable, but following intravenous transplantation significantly declined to 0.8%. (FIG. 1B)

Under αβ TCR/CsA protocol T-lymphocyte subpopulation was significantly depleted at day 7 and repopulated to pre-transplant level at day 63. After intraosseous transplantation of 70×10⁶ SCT at day 7 chimerism was 5.7% for RT1ⁿ/CD4, and increased up to 6.5% at day 63. In contrast in intravenously transplanted groups at day 7 chimerism was higher and showed 23.7% for RT1ⁿ/CD4 antigens, however declined to 0.9% at day 63. Multilineage chimerism was 75% higher in intraosseous (9.9%) when compared with intravenous (3.4%) transplant groups receiving 70×10⁶ SCT at day 63. Following intraosseous SCT under αβ TCR/CsA protocol, multilineage chimerism was 50% higher in group receiving 70×10⁶ stem cells (9.9%) compared to 35×10⁶ stem cells (4.9%). (FIG. 1C)

Conclusions: Stem cell transplantation under αβ TCR/CsA protocol showed better engraftment of donor cells following intraosseous delivery leading to development of higher level (75%) of multilineage donor-specific chimerism when compared with intravenous transplantation.

High level of chimerism in intravenously transplanted groups was transient, whereas intraosseous transplantation induced stable chimerism, which was maintained up to 63 days post-transplantation. This strategy of intraosseous stem cell transplantation under αβ TCR/CsA protocol could be applied for chimerism and tolerance induction during transplantation of composite and solid organ allografts.

### Example 2

Composite tissue allografts such as human hand comprise vascularized bone marrow as a part of allograft transplantation. This example evaluates the efficacy of vascularized bone marrow (VBMT) and hematopoietic stem cell transplantation (HSCT) on development of chimerism and induction of donor specific tolerance.

Methods: Thirty six vascularized bone marrow (femur) transplants (VBMT) containing 50×10⁶ to 70×10⁶ cells were performed across MHC barrier between Brown Norway (BN; RT1ⁿ) donors and Lewis (LEW; RT1^{l}) recipients in six experimental groups of six animals each. Isograft controls between isogenic Lewis rats included group 1—VBMT and group 2 (VBMT+HSCT) without treatment. Rejection controls between BN and LEW rats included group 3—VBMT and group 4 (VBMT+HSCT) without treatment. In treatment groups across MHC barrier (BN to LEW) group 5—VBMT and group 6 (VBMT+HSCT), allograft recipients were subjected to 7 day protocol of αβ TCR mAb/CsA therapy. Intraosseous HSC transplant (35×10⁶ cells) into the contralateral femur was performed in groups 2, 4 and 6. Flow cytometry was used for evaluation of efficacy of immunomodulation and donor specific chimerism (MHC class I-RTIⁿ) at post-transplant days 7, 21, 35, 63 and 100.

Results: Isograft controls (group 1 and 2) survived over 120 days. Non-treatment allografts showed signs of rejection (bone exposure, inflammation, infection and hair loss) between days 25 and 35 post-transplant in group 3 (VBMT), and between days 25 and 40 post-transplant in group 4 (VBMT+HSCT). Allograft recipients in treatment groups (groups 5 and 6) survived with no sign of rejection throughout the follow-up period up to 120 days. Transplantation of VBM (group 3) and VBM+HSC (group 4) without treatment resulted in development of low level donor-specific chimerism between days 7 and 35 in both T and B-lymphocyte cell populations and ranged from 1.49% to 2.35% (Table 1). In groups 5 and 6 (αβ TCR mAb+CSA protocol) flow cytometry at day 7 revealed >95% efficacy for T-lymphocyte depletion. T-lymphocytes repopulated to the pretreatment level at day 63 post-transplantation. At day 7 after transplantation high level of donor specific chimerism was observed in group 5 for RT1ⁿ/CD4+ (22.28%) and for RT1ⁿ/CD8+ (14.58%) of T cell subpopulations. In group 6 augmented with HSCT highest level of multilineage chimerism was seen at day 7 and revealed 26.47% of RT1ⁿ/CD4+ and 18.46% of RT1ⁿ/CD8+. (See Table 1.)

In both αβ TCR mAb/CSA treatment groups, T-lymphocyte chimerism decreased over time and switched to predominantly B-lymphocytes chimerism at day 21 post-transplant (Table 1). In group 5, chimerism was maintained through the B-lymphocytes, whereas in group 6 augmented with HSCT, chimerism was maintained through both T- and B-lymphocytes, with B-lymphocytes playing most important role in the chimerism maintenance (Table 1).

Conclusions: Transplantation of vascularized bone without immunosuppression provides a substantial source of bone marrow derived hematopoietic cells within its natural microenvironment, leading to development of donor specific chimerism up to 35 days post-transplant. Therapy with a 7-day αβTCR/CsA protocol facilitates development and maintenance of stable mixed multilineage chimerism up to 100 days post-transplant leading to tolerance induction in fully MHC mismatched transplants. Augmentation of vascularized bone transplant with direct intraosseous HSC transplantation allows for chimerism maintenance at 75% higher level up to 100 days post-transplant.

TABLE 1
Donor-specific chimerism during follow-up after VBMT

| | | Level of donor specific chimerism, mean value [%] | | | | |
|---|---|---|---|---|---|---|
| Experimental | Day after | | | | Total | |
| groups* | transplantation | CD4/RT1ⁿ | CD8/RT¹ⁿ | CD45RA/RT1ⁿ | number | Survival |
| VBMT | +7 | 0.58 | 0.35 | 0.84 | 1.77 | 25 to 35 |
| No treatment | +21 | 0.65 | 0.36 | 0.95 | 1.96 | |
| (group 3) | +35 | 0.78 | 0.37 | 1.20 | 2.35 | |
| | +63 | Rejected | | | | |
| | +100 | | | | | |
| VBMT + HSCT | +7 | 0.34 | 0.21 | 0.94 | 1.49 | 25 to 40 |
| No Treatment | +21 | 0.17 | 0.23 | 1.22 | 1.62 | |
| (group 4) | +35 | 1.01 | 0.4 | 0.46 | 1.87 | |
| | +63 | Rejected | | | | |
| | +100 | | | | | |
| VBMT + TCR/CsA | +7 | 22.28 | 14.58 | 4.69 | 41.55 | >120 |
| (group 5) | +21 | 1.83 | 1.30 | 7.37 | 10.5 | |
| | +35 | 0.40 | 0.60 | 12.98 | 13.98 | |
| | +63 | 0.48 | 0.42 | 1.86 | 2.76 | |
| | +100 | 0.71 | 0.67 | 1.32 | 2.70 | |
| VBMT + HSCT + | +7 | 26.47 | 18.46 | 3.12 | 48.05 | >120 |
| TCR/CsA | +21 | 0.52 | 0.29 | 7.48 | 8.29 | |
| (group 6) | +35 | 1.50 | 1.32 | 13.24 | 16.06 | |
| | +63 | 2.09 | 1.50 | 6.56 | 10.15 | |
| | +100 | 5.08 | 1.95 | 5.22 | 12.25 | |

*Isograft control group 1 and 2 not included
VBMT—Vascularized Bone Marrow Transplantation,
HSCT—Hematopoietic Stem Cells Transplantation

### Example 3

The induction of chimerism and phenotype of bone marrow (BM) cells was assessed following vascularized bone marrow (VBM) transplantation in MHC mismatched rat pairs under combined αβ T-cell receptor monoclonal antibody and cyclosporine A (αβ TCR mAb/CsA) protocol.

Method: Eighteen VBM transplantations were performed in three groups of 6 animals each. Lewis (RT1^{l}) to Lewis isotransplants were performed in Group 1 and served as isograft controls (n=6). In group 2 and 3 allotransplants across full-MHC barrier were performed from BN (RT1n) donors to Lewis recipients. Allograft rejection controls in Group 2 did not receive any treatment (n=6), whereas Group 3 allografts were treated with combined αβ TCR mAb/CsA protocol for 7 days (n=6).

Flow cytometry (FC) was used for evaluation of immunomodulation of T-lymphocytes and donor-specific chimerism for MHC class-I-RT1ⁿ antigens at day 7. Phenotype of bone marrow (BM) cells in grafted and host bone was assessed at day 7.

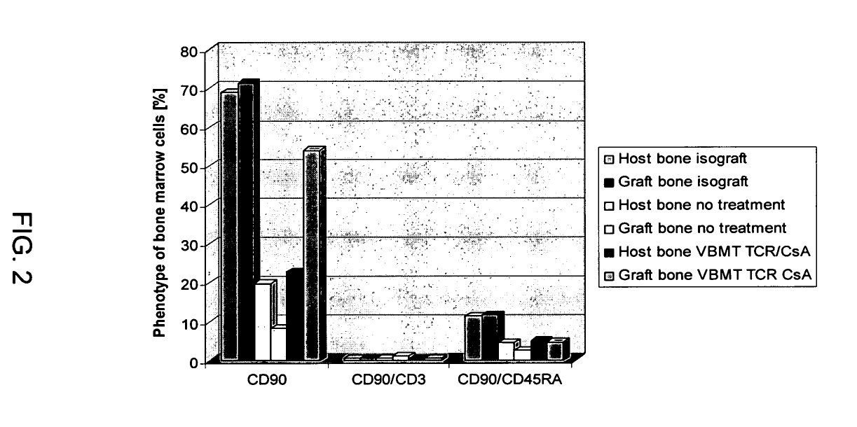

Results: In isograft controls at day 7, no significant differences were observed between total number of bone marrow cells in grafted and host bones (47.5×10⁶ vs 43.75×10⁶ cells respectively). FC analysis almost revealed equal number of stem sells in isograft and host bones at day 7 (33.9×10⁶ vs 30.3×10⁶). Double staining showed below 1% of CD90/CD3 cells and 11% of CD90/CD45RA cells in both grafted and host bone. (FIG. 2)

In VBM transplants treated with combined αβ TCR/CsA protocol, total number of BM cells in grafted bones ranged from 18.75×10⁶ to 43.75×10⁶ cells, whereas in host bones ranged from 31.25×10⁶ to 41.25×10⁶ cells. Analysis of BM cell phenotype at day 7 revealed 22.8%-74.1% CD90⁺ cells in grafted bone and 13.2%-34.2% in host bone. Double positive CD90⁺/CD45RA⁺ cells ranged from 8.1% to 12.6% in grafted bone and from 2.4% to 6.7% in host bone. The percentage of CD90⁺/RT1ⁿ⁺ cells in host bone ranged from 0.3% to 4.0% indicating migration of donor cells to the recipient compartment.

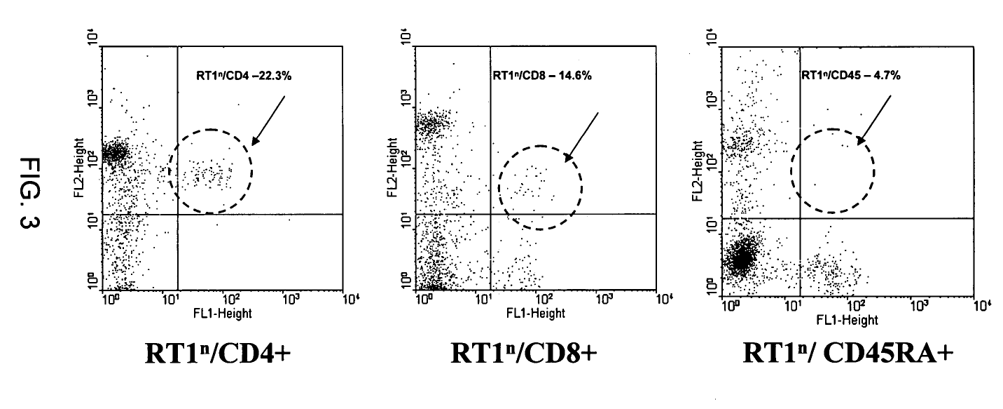

At day 7 in the allograft rejection control group, the chimerism level was below 1% in T- and B-lymphocyte populations. In the VBM transplantation group treated with αβ TCR/CsA, T-cell population was significantly depleted at day 7 (<95%). Multilineage donor-specific chimerism revealed 22.3% CD4/RT1ⁿ and 14.6% CD8/RT1ⁿ T-lymphocyte subpopulation and 4.7% CD45RA/RT1ⁿ of B-lymphocytes. (FIG. 3)

Conclusion: Vascularized bone marrow allograft transplantation allowed for successful chimerism induction under αβ TCR mAb/CsA protocol. Interestingly, VBMT was characterized by over 50% higher engraftment of donor specific B-cell lineage (CD90⁺/CD45RA⁺).

### Example 4

In this example, induction of chimerism was assessed following vascularized bone marrow transplantation across MHC barrier.

Method: BN (RT1ⁿ) rats were used as donors, and Lewis (RT1^{l}) rats were used as recipients of vascularized femoral bone transplants. A total of 18 transplants were divided into three groups, of six animals each group. Group I—isograft controls (n=6), Group II—allograft rejection controls without treatment (n=6), and Group III—allografts treated with αβ TCR and CsA for seven days (n=6).

Flow cytometry (FC) was used to assess the efficacy of immunodepletion of T-lymphocytes and donor-specific chimerism for MHC class I-RT1ⁿ CD4, CD8, and CD45RA lymphocytes at day 7, 21, 35, 63 and 100 post-transplantation.

Histological H&E stained femur samples were assessed for bone marrow viability and signs of rejection.

Results: The animals in the group I survived indefinitely. Group II allograft rejection controls showed signs of wound infection, bone exposure and bone necrosis at day 21 post-transplantation. All animals in the group III treated with αβ TCR and CsA for seven days survived without any sign of graft versus host disease for over 100 days.

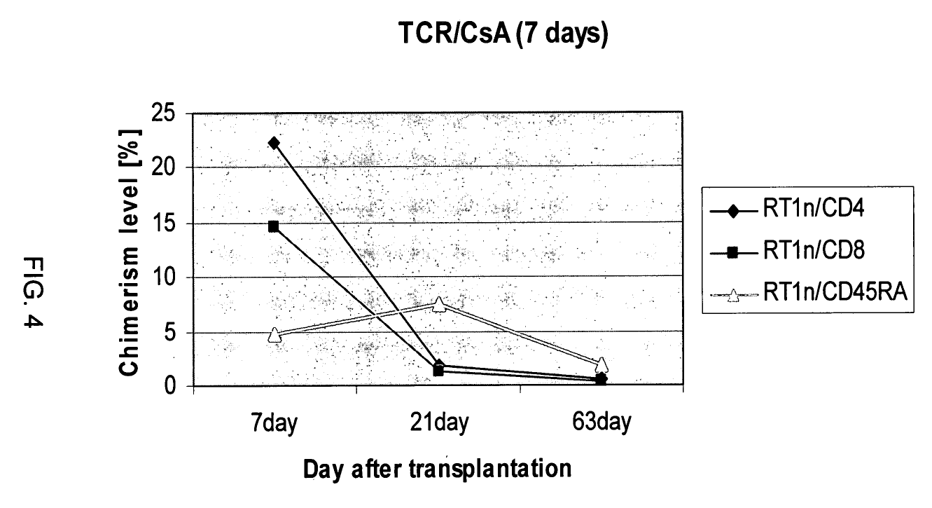

FC analysis, in the group III, revealed 92% of T-lymphocyte depletion at day 7 and repopulation to preoperative values at day 63. T-cell chimerism was 22.3% CD4/RT1ⁿ and 14.6% CD8/RT1ⁿ at day 7, and 1.9% at day 63. At day 21 T-cell chimerism decreased and switched to B-cell chimerism of 7.4% CD45RA/RT1ⁿ and remained at 1.9% at day 63. (FIG. 4)

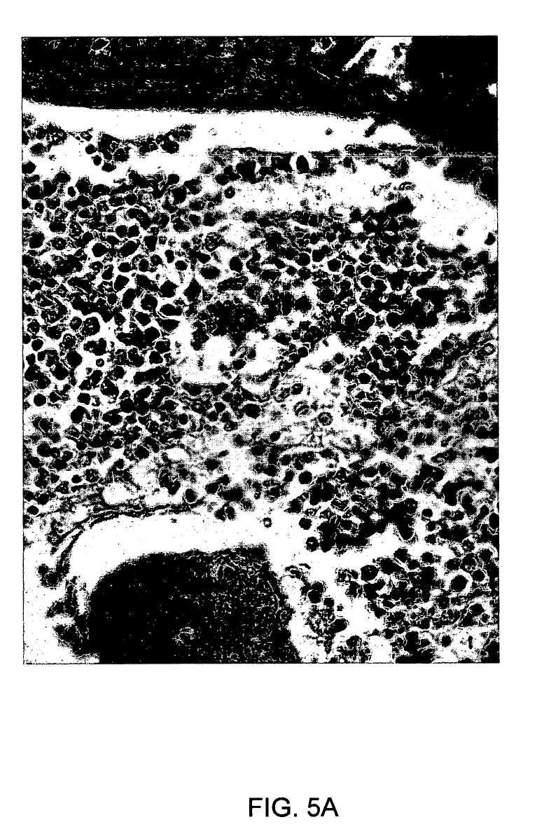

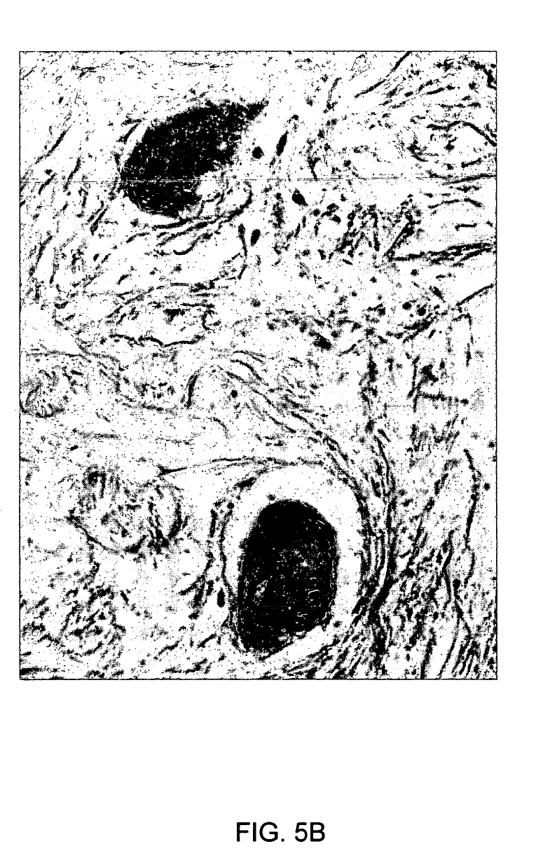

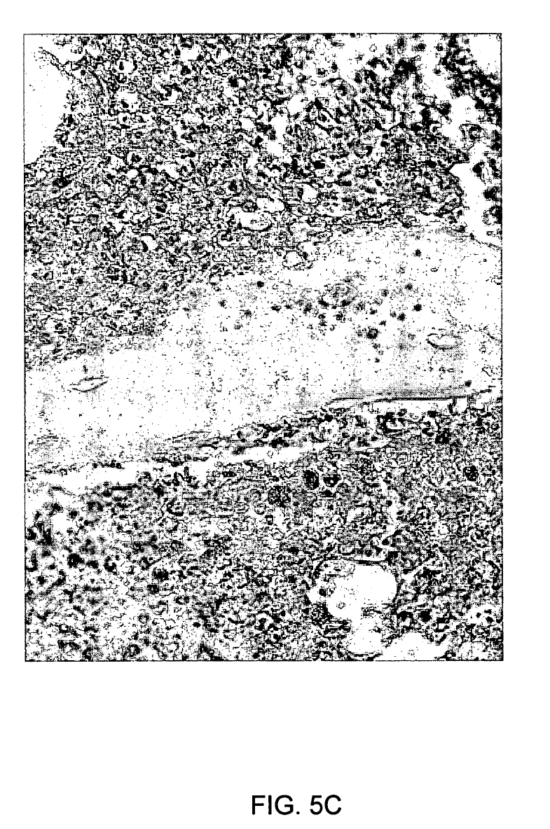

Histological examination revealed normal architecture and viability of bone marrow at day 7 in isograft control (FIG. 5A), and in αβ TCR and CsA treatment group (FIG. 5B), and grade II signs of rejection in non-treated transplants. (FIG. 5C)

Conclusion: VBMT under αβ TCR and CsA protocol resulted in chimerism induction and maintenance for over 100 days post-transplant. Chimerism was characterized by switch from T-cell to B-cell lineage at day 21 post-transplant.

### Example 5

This example evaluates the efficacy of vascularized bone marrow (VBM) and hematopoietic stem cell (HSC) transplantation on induction of chimerism and donor specific tolerance.

Methods: Twenty four vascularized bone marrow (femur) transplants (VBMT) were performed across MHC barrier between BN (RT1ⁿ) donors and Lewis (RT1^{l}) recipients in six experimental groups of six animals each. In-group I, VBM transplantations were performed and no treatment was given. In-group II, VBM transplants were treated with anti-αβ TCR mAb/CsA for 7-days. In-group III, VBMT was augmented by hematopoietic stem cells transplantation (HSCT), and no treatment was given. In-group IV, augmented with HSC vascularized bone marrow transplant were treated with αβ TCR+CSA for 7-days. Efficacy of immunosuppression and chimerism were evaluated by flow cytometry in peripheral blood at post-transplant days 7, 21 and 35 of T- (CD4, CD8) and B-lymphocytes (CD45RA).

Results: All animals survived without sign of graft versus host disease. Flow cytometry analysis, at day 7, revealed that VBMT augmented with stem cells had the highest level of multilineage chimerism for 31.5% and 21.9% of RT1ⁿ/CD4+ and RT1ⁿ/CD8+ cells respectively, while they were 22.3% and 14.6% in animals with VBMT. At day 35 post-transplant, T cell chimerism declined <1% in animals with VBMT, and was 1.2% and 1.36% in animals augmented with HSC. Chimerism in B-lymphocytes, however, increased gradually, and showed 4.7% vs 12.5% RT1ⁿ/CD45RA⁺ in animals with VBMT, and 3.66% vs 11.38% in animals with augmented VBMT at day 7 and 35 respectively. In non-treated groups, the level of chimerism was as low as <1% at day 7.

Conclusions: VBMT under short-term αβ TCR+CsA protocol was found to be effective in induction of donor specific chimerism. Interestingly, chimerism was achieved for the T-lymphocyte population in early post-transplant period and switched to B cell chimerism at day 35 post-transplant. Addition of HSC transplantation significantly increased donor specific chimerism.

### Example 6

This example evaluates whether the hind limb allograft is a stronger bone marrow delivery source when compared to isolated vascularized bone marrow transplantation (VBMT) for chimerism and tolerance induction.

Methods: BN (RT1ⁿ) rats were used as donors, and Lewis (RT1^{l}) rats were used as recipients for composite tissue allotransplantations (CTA). Thirty six transplanted animals were divided into six experimental groups of 6 animals each. Group I isograft limb control, group II isograft VBMT control, group III limb allograft and group IV VBMT allograft rejection controls without treatment. Treatment groups αβ TCR+CSA 7 days included group V—limb allograft and group VI—VBMT.

Flow cytometry was used for evaluation of immunomodulation (CD3⁺, CD4, CD8 T-lymphocyte level) and of donor specific chimerism in peripheral blood for MHC class I-RTIⁿ specific antigens at post-transplant days 7, 21 and 35, and 63.

Following H&E staining, limb and femur samples were graded histologically for evidence of sign of rejection.

Results: Animals in groups I and II showed indefinite survival. In control group III limb allografts rejected between 5-7 days post-transplant. In group IV, animals began showing rejection signs such as wound infection, bone exposure and bone necrosis at day 21 post-transplant. All animals treated with a αβ TCR+CSA 7-day protocol survived without any sign of graft versus host disease.

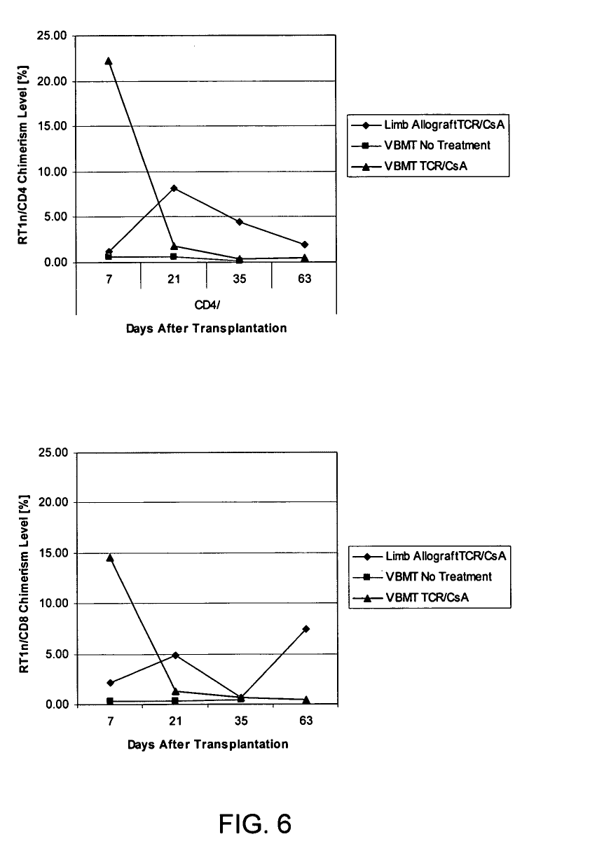

Flow cytometry analysis revealed that T-lymphocyte population (CD3⁺) was significantly depleted at day 7 in groups V and VI under αβ TCR+CSA protocol. At day 63 post-transplant repopulation of T cells to the pre-transplant level was seen. At day 7, treated limb allografts showed 1.26% of RT1ⁿ/CD4⁺ and 2.2% of RT1ⁿ/CD8⁺ of chimerism, and this level increased to 1.93% of RT1ⁿ/CD4⁺ and 7.43% RT1ⁿ/CD8⁺ in T-cell subpopulations, at day 63. In contrast, in VBMT chimerism at day 7 was higher and reveled 22.3% of RT1ⁿ/CD4⁺ and 14.6% of RT1ⁿ/CD8⁺ cells; however, it declined to below 1% at day 63. (FIG. 6)

Histology confirmed graft rejection in non-treated allograft transplants, and low-grade inflammation in treated allografts.

Conclusions: Short protocol of αβ TCR+CsA proved to be successful in chimerism induction in both limb and vascularized bone marrow allograft transplantation models. Interestingly high level of VBMT chimerism was only transient, whereas limb allografts showed lower but stable level of chimerism, which correlated with indefinite graft survival.

### Example 7

Extended Survival of Allogeneic Skin Transplants in Recipients under a Combined CsA/αβ TCR mAb Immunosuppressive Treatment Protocol and Direct Transplantation of Crude Donor Bone Marrow into Recipient Bone Marrow Cavity

In this example, 43 skin graft transplantations were performed in 9 animal groupings, described below, between isogeneic [Lewis to Lewis (LEW, RT-1^{L})] and semi-allogeneic [Lewis x Brown Norway (LBN→F1, RT-1^{l+n}) to Lewis] rat strains under anti-αβ-TCR mAb and CsA treatment. The allogeneic skin graft recipient also received a crude bone marrow transplantation from the same donor into a recipient bone marrow cavity. The use of combined protocol of CsA/-TCR mAb and the crude bone marrow transplantation resulted in the extension of skin allograft survival up to 65 days after cessation of the immunosuppressive treatment (p<0.05).

The following animals, reagents, assays and techniques were employed.

I. Animals

Inbred male rats weighting 150-175 grams were purchased from Harlan Sprague-Dawley, Indianapolis, Ind.). Lewis rats (LEW) served as the recipients of skin and crude bone marrow allografts from Lewis-Brown Norway donors (LBN). The animals were caged at room temperature on a 12-hour light/dark cycle with free access to food and water. All animals received humane care in compliance with the Guide for the Care and Use of Laboratory Animals published by the National Institute of Health in the facility accredited by the American Association for the Accreditation of Laboratory Animal Care.

II. Transplantation Techniques

Skin grafting and crude bone marrow transplantation were performed at the same session from the same donor. Intraperitoneal pentobarbital (50 mg/kg) was used as an analgesic during the transplantation procedure.

A. Skin Grafting Transplantation Technique

Skin grafting was performed according to the technique described by Billingham (Billingham R E. and P. B. Medawar. J. Ex. Biol. 1951; 28: 385-402). Briefly, full thickness skin grafts 16 mm in diameter were taken from the donors. Graft beds were prepared by excising 18 mm circles on the lateral dorsal thoracic walls of the recipients. Care was taken to remove perniculous carnosum from the grafted skin. Both sides of the thoracic wall were used for allogeneic grafts and the mid-sternum was used for syngeneic grafts. All grafts were separated by a 10 mm skin bridge. A standard compressive dressing and adhesive bandage was used for 7 days.

B. Crude Bone Marrow Transplantation Technique

Donor-origin bone marrow was transplanted in its crude form containing all natural microanatomical components of the bone marrow including pluripotent stem cells and progenitor cells and extracellular matrix.

(a). Harvesting of the crude bone marrow from the donor: The metaphyseal region of the right tibia was approached from an anterior incision. Next, on the anteromedial cortex of the tibia, a 3 mm window was created using a 1/32-inch drill. Following decortication, the contents of the metaphyseal part of the bone were removed with a bone curette and placed in a container cooled in ice. Next, the bone rongeur was used to harvest intramedullary content along the diaphysis. The weight of the harvested BM was measured before transplantation.

(b). Transplantation of the crude BM to the recipient: The recipient's tibia was opened in a similar fashion as the donor's bone, exposing the anteromedial surface of the right tibia, followed by creation of the cortical window. The recipient's cancellous bone and content of the BM cavity were totally removed to create an appropriate “space” for the BM engraftment. The harvested bone marrow of donor origin was next packed into the “empty” medullary cavity of the recipient's tibia and sealed with bone wax.

III. Immunosuppressive Treatment Protocols

Treatment protocols included monotherapy with CsA alone or anti-αβ TCR monoclonal antibody (anti-αβ TCR mAb) alone, or a combination of CsA and anti-αβ TCR monoclonal antibody (CsA/αβ TCR mAb). Both CsA and anti-αβ TCR mAb were administered 12 hours before transplantation and continued up to 7 days or 35 days in the combined therapy group.

Cyclosporine A (Sandoz Pharmaceutics Inc., East Hanover, N.J.) was dissolved daily in PBS (Fisher Scientific, Pittsburgh, Pa.) to a concentration of 5 mg/ml and administered subcutaneously (s.c.) to recipient animals. Animals under CsA treatment received a dose of 16 mg/kg/day (s.c.) administered 12 hours before transplantation and daily thereafter for the first week, 8 mg/kg/day during the second week, 4 mg/kd/day for the third and fourth week, and 2 mg/kg/day for the fifth week. Intraperitoneal (i.p.) injection of anti-αβ TCR mAb (clone R73, Pharmingen, San Diego, Calif.) (250 μg) was administered 12 hours before transplantation, and daily thereafter for the first week. The dosage of anti-αβ TCR mAb was then tapered to 50 μg at the end of the first week and was given every 2 days during the second week and every 3 days during the last 3 weeks.

The 7-day protocol was similar. Animals under CsA treatment received a dose of 16 mg/kg/day (s.c.) administered one hour or 12 hours before transplantation (when semi-allogeneic transplants were performed) and daily thereafter for 7 days. Intraperitoneal injection of anti-αβ TCR mAb (250 μg) was administered one hour or 12 hours before transplantation (when semi-allogeneic transplants were performed), and daily thereafter for 7 days.

In each of the foregoing protocols, when fully allogeneic transplants were performed, both the CsA and anti-αβ TCR mAb treatments were initially administered at the time of transplantation, at the time of clamp release.

IV. Treatment Groups

The treatment groups, treatment protocols, and graft survival are illustrated in Table 1. Multiple trials were performed according to the protocols. The immunosuppressive treatment was administered using the 35 protocol or the 7 day protocol. The amount of crude donor bone marrow transplanted ranged from 20 mg to 100 mg.

The following treatment groups were employed in one series of transplants between semi-allogeneic donors/recipients:

Group 1: Skin Isograft Control (n=6): Skin grafts were transplanted between LEW rats without immunosuppressive treatment before or after transplantation.

Group 2: Skin Allograft Control (n=6): Skin allografts were transplanted from LBN donors to LEW recipients without immunosuppressive treatment before or after transplantation.

Group 3. Skin and Bone Marrow Allograft Control (n=6): Both the skin and the crude bone marrow were transplanted from donor LBN to recipient LEW rats without immunosuppressive treatment before or after transplantation.

Group 4. Skin Allografts+CsA (n=3): Skin allografts were transplanted from LBN donor to LEW recipients. Animals in this group received 5 weeks of CsA treatment following skin transplantation.

Group 5. Skin Allografts+anti-αβ TCR mAb (n=3): Skin allografts from LBN donors were transplanted to LEW recipients. Animals received 5 weeks of anti-αβ TCR mAb treatment following skin transplantation.

Group 6. Skin Allograft+CsA+anti-αβ TCR mAb (n=5): Skin allografts from LBN donors were transplanted to LEW recipients. Animals received 5 weeks of combined CsA and anti-αβ TCR mAb treatment following skin transplantation.

Group 7. Skin Allograft+BM+CsA (n=5): Skin and the crude bone marrow from the same LBN donors were transplanted to LEW recipients. The recipient received 5 weeks of CsA treatment following transplantation.

Group 8. Skin Allograft+BM+anti-αβ TCR mAb (n=4): Skin and the crude bone marrow from the same LBN donors were transplanted to LEW recipients. The recipient received 5 weeks of anti-αβ TCR mAb treatment following transplantation.

Group 9. Skin Allograft+BM+CsA+anti-αβ TCR mAb (n=5). Combined CsA and anti-αβ TCR mAb protocol was applied for 5 weeks following the skin and the crude bone marrow allograft transplantation from the same LBN donors to LEW recipients.

V. Clinical Assessment of Allograft Rejection

The physical signs of skin allograft rejection, such as erythema, edema, loss of hair, scaling of the skin, and desquamation were evaluated on a daily basis. Rejection was defined as the destruction of over 80 percent of the graft.

VI. Assessment of Graft Versus Host Disease (GVHD)

All animals were evaluated for the appearance of GVHD. The clinical criteria, such as diffuse erythema (particularly of the ear), hyperkeratosis of the foot pads, dermatitis, weight loss, generalized unkempt appearance, or diarrhea were monitored daily. An animal was considered to exhibit acute GVHD if at least four of the above signs were observed. The diagnosis of GVHD was confirmed by routine hematoxylin and eosin histologic staining performed on formalin-fixed skin, tongue, liver and small intestine samples collected at the time of occurrence of the first signs of GVHD. Grading of GVHD was performed in blinded fashion according to previously described histologic criteria (Sale, G. E. et al Am. J. Surg. Pathol. 1979; 3: 291; Saurat, J. H. et al. Br. J. Dermatol. 1975; 93: 675) and were assessed by a histopathologist.

VII. Flow Cytometry Analysis

Flow cytometry (FC) analysis was performed according to the manufacturer's protocol (Becton Dickinson, San Diego, Calif.) with minor modifications. The blood samples of transplant recipients were collected into heparinized tubes on the following days post-transplantation: 0, 7, 21, 35, 63 and at the time of initial signs of clinical rejection. The peripheral blood mononuclear cells (PMBC) were incubated for 20-30 minutes in the dark at room temperature with 5 μL of a mixture of mouse anti-rat monoclonal antibodies conjugated with fluorescein isothiocyanate (FITC) or phycoerythrin (PE) against CD4⁻FITC (Clone OX35), CD8a⁻PE (Clone OX8), αβ TCR⁻FITC (Clone R73), CD45RA⁻PE (Clone OX-33). After incubation, samples were resuspended in FACS Lysing solution (Becton Dickinson), incubated for 20 minutes in the dark at room temperature, centrifuged at 1500 rpm for 5 minutes, washed twice with washing buffer (phosphate-buffered saline, PBS, without Mg⁺⁺, Ca⁺⁺, 0.1% bovine serum albumin, 0.05% NaN₃), fixed with 2% paraformaldehyde solution, covered with aluminum foil and stored at 4° C. until flow cytometry assessment.

VIII. Donor-Specific Chimerism Evaluation by FC

For the determination of donor-specific lymphoid chimerism in the peripheral blood of recipients, combinations of mouse anti-rat CD4⁻PE or CD8a⁻PE with RT-1ⁿ (Brown Norway MHC class I, Clone MCA-156, Serotec, Kidlington, UK) were applied. For the donor-derived, RT-1ⁿ-positive staining cells, purified anti-rat CD-32 (FcyII Block Receptor) antibody (1:20) was added first to block the Fc-mediated adherence of antibodies. After 3-4 minutes pre-incubation, samples were further incubated with 5 μL of mouse anti-rat RT-1ⁿ for 30 minutes at 4° C. Then samples were washed twice in washing buffer and stained with goat anti-mouse FITC conjugated IgG (rat adsorbed; Serotec). This was followed by the incubation with CD4⁻PE or CD8⁻PE conjugated mouse anti-rat monoclonal antibody. After incubation, samples were processed as described above. The negative control included isotype-matched antibodies and/or PBS-incubated samples. FC analyses were performed on 1×10⁴ mononuclear cells by using FACS Scan (Becton Dickinson) and CellQuest software.

IX. Statistical Evaluation

Treatment groups were compared on survival using the log-rank test, and survival times were estimated using the Kaplan-Meier method. The level of chimerism, and efficacy of the immunosuppressive treatment were compared by the independent samples t test. Differences were considered statistically significant at p<0.05.

The Survival of the Skin Allografts. The survival of skin allografts in each transplant group is presented in Table 1. The combined protocol of CsA and anti-αβ TCR mAb applied to the recipients of skin allografts without bone marrow significantly extended allograft survival when compared to the allograft controls without treatment (p<0.05). However, following cessation of CsA/αβ TCR mAb therapy, all allografts were rejected within 20 days (p<0.05). It was demonstrated that monotherapies, combined with crude donor bone marrow transplantation, resulted in extended survival up to 21 days (under CsA) and up to 10 days (under anti-αβ TCR mAb). Monotherapy with CsA alone extended skin transplant survival over 7 days, whereas monotherapy with anti-αβ TCR mAb alone resulted in rejection at the same time as the rejection controls without treatment. When skin allograft transplantation and the crude bone marrow transplantations were performed in one procedure, the skin allograft transplant survival was extended by over 60 days (p<0.05) after cessation of the CsA/αβ TCR mAb protocol.

Extended skin allograft survival was achieved in animals receiving crude bone marrow under short term (7 days only) and longer term (5 weeks) CsA/αβ TCR mAb treatment protocol.

Determination of GVHD. None of the allogeneic transplant recipients showed clinical signs of GVHD.

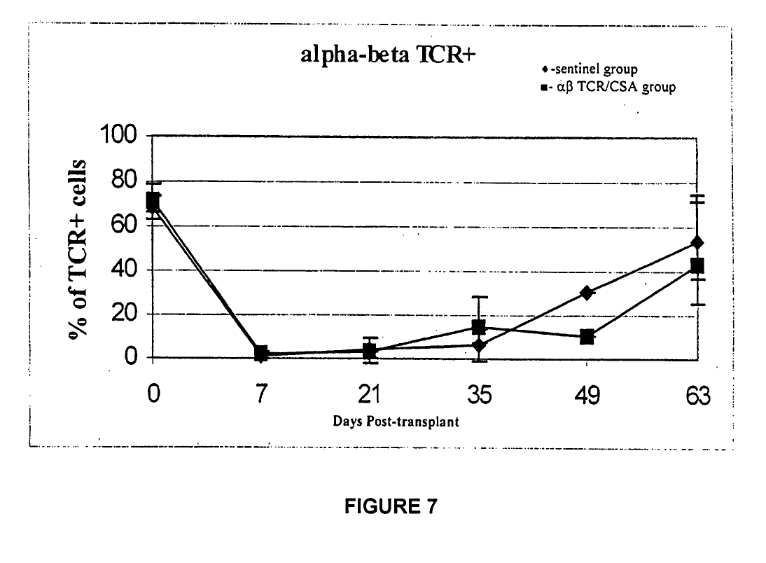

Flow Cytometry Analysis of In Vivo Depletion of αβ TCR⁺ Cells. As illustrated in FIG. 7, FC determination of αβ TCR expression on lymphocytes harvested from sentinel (untransplanted animals treated with CsA/αβ TCR mAb) and transplanted animals treated with CsA/αβ TCR mAb showed >90% depletion of the αβ TCR⁺ cell population 7 days after immunosuppressive treatment cessation (day 42 post-transplantation). Repopulation of αβ TCR⁺ cell populations to the pre-transplantation level was observed 28 days after cessation of the immunosuppressive treatment (day 63 post-transplantation). The population of CD4⁺ and CD8⁺ cells in the peripheral blood of the sentinel and transplanted recipients was significantly reduced at day 7 (75% of CD4⁺ and 55% of CD8⁺) and a gradual repopulation of both cell subpopulations was seen at day 63 (data not shown).

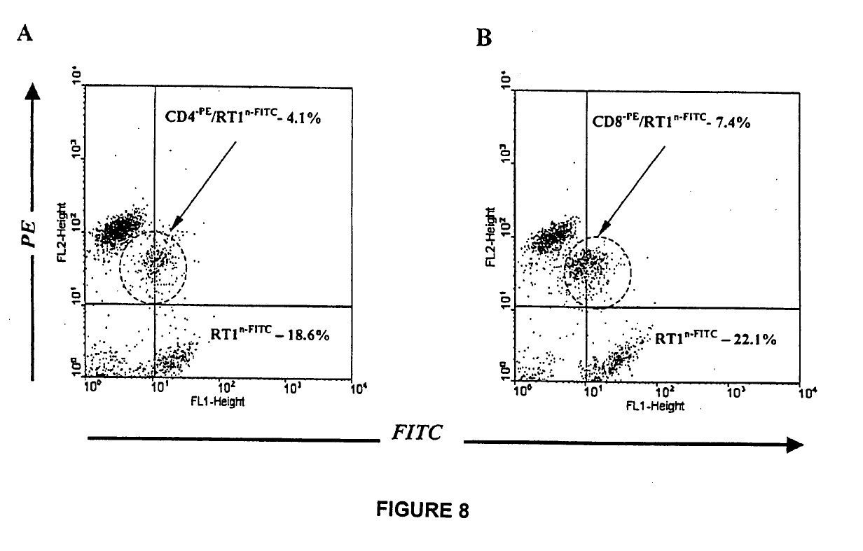

Mixed Donor-Recipient Chimerism in Recipients of Crude Donor Bone Marrow. Two-color flow cytometry analysis of the donor-specific macro-chimerism was performed on PBMC of recipients of crude donor bone marrow and skin allografts. The dot-plot results of the lymphoid cell subpopulations obtained from quadrangle analytical gates at day 65 after allotransplantation demonstrated the presence of the multilineage donor-specific chimerism ranging from 18-29% (RT-1ⁿ positive cells). Examination of two-color stained RT-1ⁿ-FITC/CD4⁻PE and RT-1ⁿ-FITC/CD8⁻PE peripheral lymphocytes revealed 4.1% and 7.4%, respectively, of double positive CD4 and CD98 T cell subpopulations, illustrated in FIGS. 8A and 8B, respectively. The expression of RT-1ⁿ antigen on non-CD4 and non-CD8 positive T cell populations suggests the existence of donor/recipient chimeric residents not evaluated with the PE-conjugated mAb against surface specific antigen (B-lymphocytes and monocytes). Therefore, the donor crude bone marrow transplant inoculated directly into the recipient's bone marrow cavity allowed for optimal engraftment, repopulation and subsequent trafficking outside the bone marrow cavity into the periphery, resulting in donor specific chimerism.

In trials employing different amounts of crude donor bone marrow ranging from 20 mg to 100 mg, it was found that chimerism and extended allograft survival was achieved when the weight of transplanted crude bone marrow was greater than about 50 mg.

### Example 8

Enhancement of CTA Survival by Intraosseous Delivery of Donor-Derived Stem Cells and Progenitor Cells

This example illustrates that increases in the level of hematopoietic chimerism can improve the survival and maintenance of CTA transplants without immunosuppressive therapy. The exemplary design included transplantation of the rat hindlimb allograft (LBN to LEW) concomitant with the direct intraosseous transplantation of bone marrow stem and progenitor cells isolated from the same donor. The following techniques and treatment regimens were employed.

I. Hindlimb Transplantation Technique

Transplantations of hindlimbs between donor and recipient were performed under pentobarbital (50 mg/kg intraperitoneal) anesthesia using a standard microsurgery procedure (Press, B. H. J. et al. 1986. Ann. Plast. Surg. 16: 313-321). Briefly, a circumferential skin incision was made in the proximal one third of the right hindlimb. The femoral artery and vein were dissected, clamped, and cut proximal to the superficial epigastric artery. The femoral nerve was dissected and cut 1 cm distal to the inguinal ligament. The biceps femoris muscle was transected to expose the sciatic nerve. The nerve was then cut proximal to its bifurcation.

The donor was prepared in a similar way. The right hindlimb was amputated at the midfemoral level. The donor limb was attached to the recipient limb by a 20-gauge intramedullary pin and a simple cerclage wire. All large muscle groups were sutured in juxtaposition. The iliac vessels of the donor and femoral vessels of the recipient were anastomosed under an operating microscope with 10-0 sutures by using a standard end-to-end microsurgical anastomosis technique. The femoral and sciatic nerves were repaired by using a conventional epineural technique with four 10-0 sutures.

II. Purification of CD90⁺ Stem and Progenitor Bone Marrow Cells

Bone marrow cells were isolated from donors using flushing methods. Briefly, freshly isolated femur and tibia were washed with sterile, cold PBS (without Mg⁺⁺ and Ca⁺⁺) supplemented with 1.0% bovine serum albumin (BSA). Two contralateral ends of the bones were cut and residual bone marrow cells were flushed out from the bone marrow cavity using a syringe-based pump system. After lysis with NH₄Cl/TRIS sterile hemolytic buffer for 5 minutes, nucleated marrow cells were then washed (PBS, 1.0 BSA) twice and counted to obtain a final concentration of 1×10⁶ nucleated cells/ml.

For isolation of the CD90⁺ cells, isolated nucleated bone marrow cells were incubated with FITC conjugated mouse anti-rat CD90 mAb (OX-7, Pharmingen) for 30 minutes in the dark at 4° C. After incubation, samples were washed twice with washing buffer, incubated for 30 minutes in the dark at 4° C. with magnetic beads-conjugated mouse anti-FITC mAb, washed, and placed into MiniMACS separation columns (Miltenyi Biotec, Auburn, Calif.). The CD90⁺ cells were collected and their viability and number assessed by counting of the cells incubated with trypan blue. The assessment of the efficacy of purification of the MACS positively selected cells was accomplished by FC evaluation of the level of double positive CD90^{FITC} (Clone OX7)/RT-1ⁿ cells.

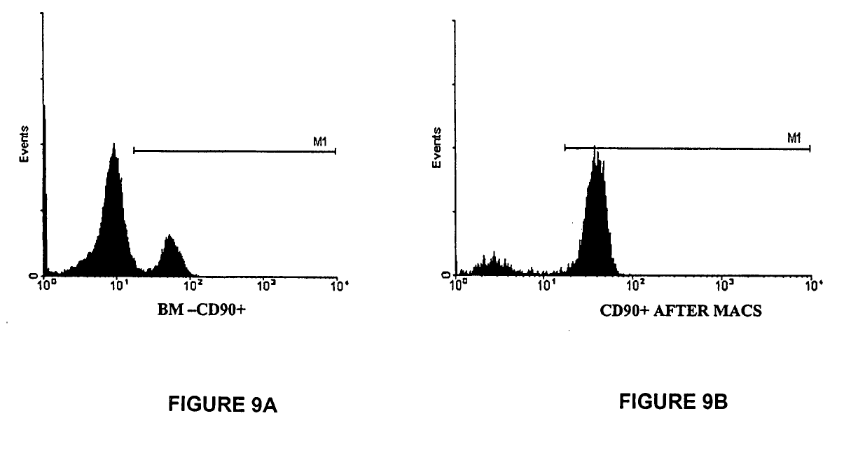

FIG. 9 illustrates FC analysis of the CD90⁺ antigen expression on the surface of stem and progenitor cells from donor bone marrow before and after selection using a magnetic MiniMACS separating system showed CD90 cells as the small peak (FIG. 9A), and the remainder of the bone marrow nucleated cells as the larger peak, prior to selective separation of the CD90⁺ cells from a suspension of bone marrow cells. The CD90⁺ cells comprised about 22% of the bone marrow nucleated cells. The purity of separation of the CD90⁺ cells (FIG. 9A, large peak) was as follows: Less than 5% were CD90⁻. Over 95% of analyzed cells expressed the CD90 antigen, indicating high efficacy of the selection.

III. Intraosseous Injection of Donor Derived Stem and Progenitor Cells

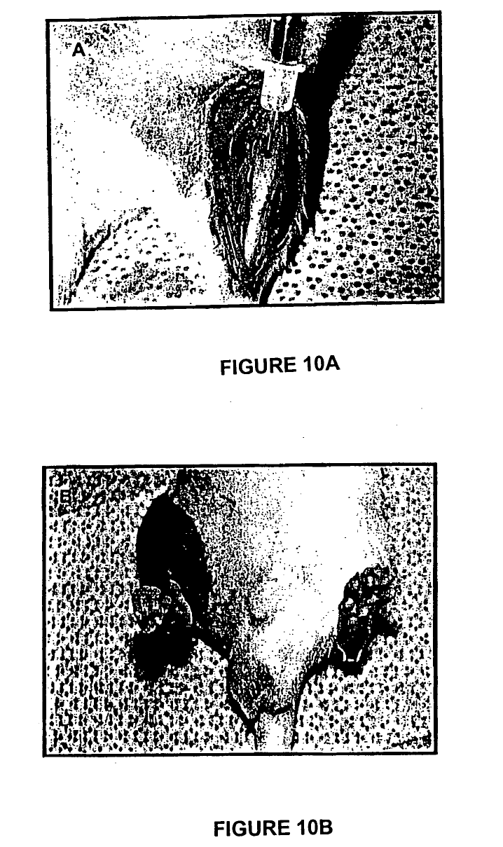

A 50 μL high-purity suspension containing 8-12×10⁵ CD90⁺ stem and progenitor cells was injected directly into the bone marrow cavity of the recipient's contralateral tibia just before transplantation of the opposite hindlimb. No immunosuppressive protocol was given. FIG. 10A illustrates injection of the stem and progenitor cells into the bone marrow cavity of the recipient's left tibia. Following injection, the right hindlimb from the same donor was transplanted to the recipient. (FIG. 10B)

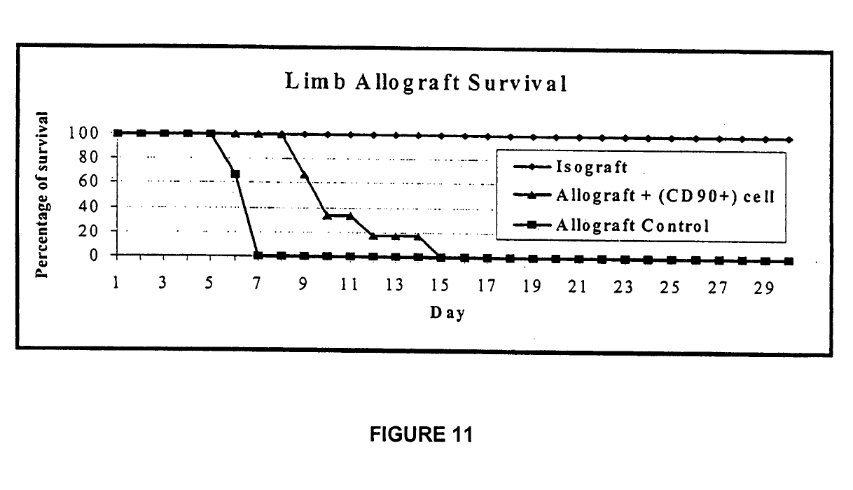

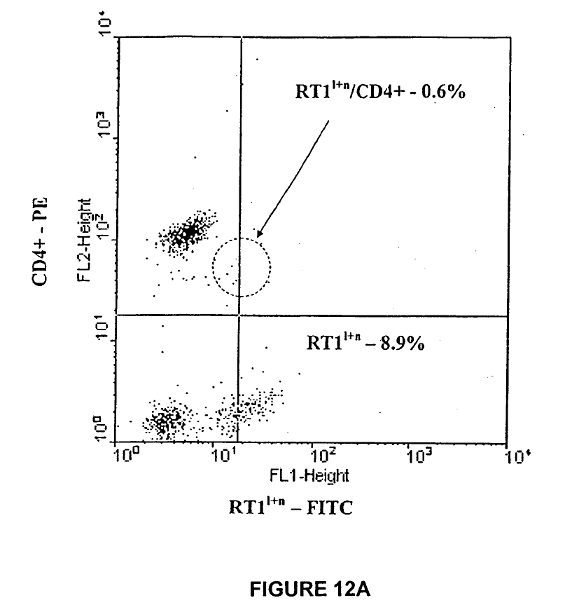

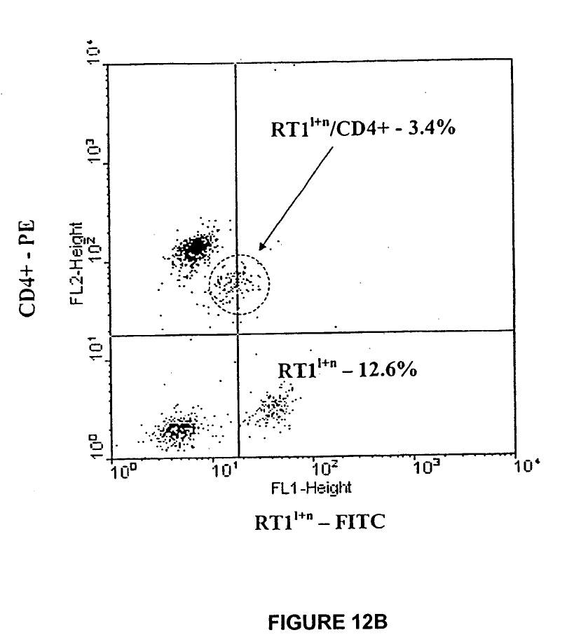

Survival of Limb Allografts Following Pre-operative Intraosseous Injection of Donor Stem and Progenitor Cells. As shown in the limb allograft survival chart illustrated in FIG. 11, recipients receiving the allograft and also receiving intraosseous injection of donor CD90⁺ stem and progenitor cells, had a significant biological extension (up to 15 days) of limb allograft survival without immunosuppressive therapy (p<0.05), compared to recipients receiving the allograft only. This survival extension correlates with a transient chimerism level of 3.4% of CD4⁺ T cells of donor origin in the peripheral blood of the recipients, illustrated by the FC analysis shown in FIG. 12. A two-color FC analysis at the 14^{th} day after limb allograft transplantation revealed transient chimerism in the allograft rejection control group without treatment (0.6%, FIG. 12A) and a higher level (3.4%, FIG. 12B) of double positive RT-1^{l+n}/CD4⁺ chimeric cells in the peripheral blood of the limb recipients treated with the intraosseous injection of the CD90⁺ stem and progenitor cells at the time of transplantation.

### Example 9

This example illustrates a comparison of the level of donor/recipient chimerism in the hindlimb transplantation model, after intraosseous transplantation of donor stem and progenitor cells or intravenous injection of the same number of donor stem and progenitor cells.

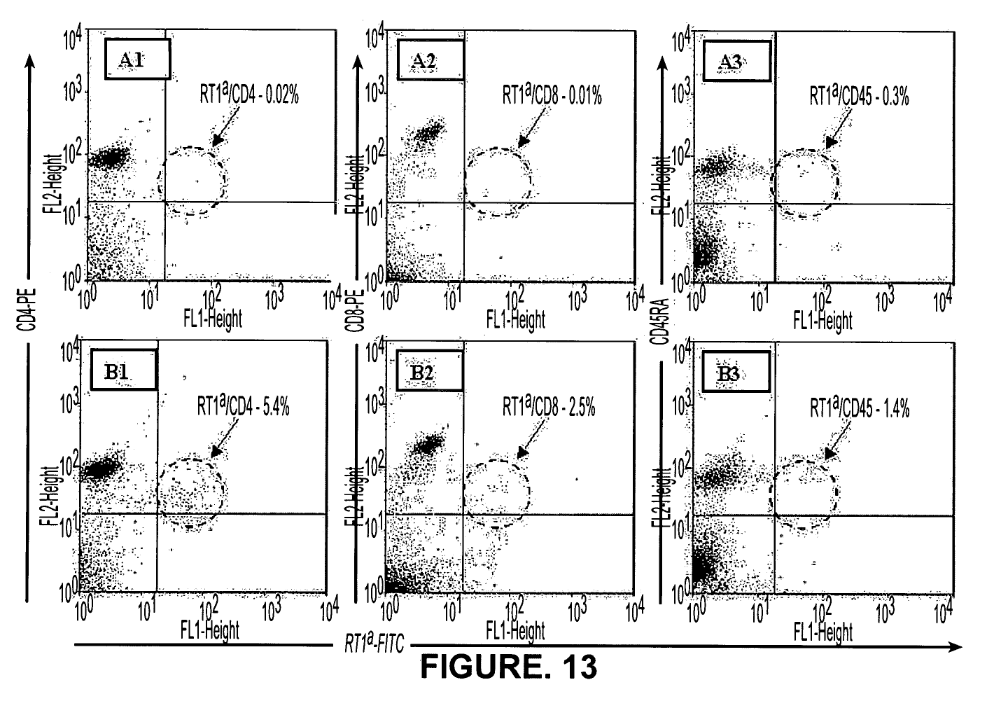

Intraosseous Transplantation of Donor Stem and Progenitor Cells Produces Long Term Donor-Specific Chimerism. In a further example, 50 μL of a high purity (>95%) suspension containing 35-40×10⁶ donor stem and progenitor cells were obtained as described above. The same number (35-40×10⁶) of cells was injected intravenously into the epigastric vein in one group of recipient rats and directly into the tibial bone marrow cavity in the another group of recipient rats. Hindlimb transplants were then performed as described above. At day 35 post-transplant, flow cytometry revealed high levels of multilineage donor-specific lymphoid chimerism (FIGS. 13B1-13B3) in the peripheral blood of the recipients receiving direct intraosseous donor stem and progenitor cells at the time of transplantation, which was maintained over 35 days (still under evaluation). In contrast, intravenous injection of the donor stem and progenitor cells at the time of transplantation resulted in low-level, transient (up to 5 days) chimerism (FIGS. 13A1-13A3). These results confirm the importance of the microanatomic environment and stromal compartment in the efficacy of stem and progenitor cell engraftment. The results show that direct delivery of the stem and progenitor cells into the bone marrow cavity results in increased efficacy of donor cell engraftment and augmentation of mixed chimerism.

### Example 10

Chimerism in Vascularized Skin/Vascularized Bone Transplantation. A vascularized embodiment of CTA comprising vascularized skin with subcutaneous fat (VS), and vascularized bone (VB) with cartilage and bone marrow was employed. The allograft transplantations were carried out across MHC semi-mismatched donors and recipients (LBN; RT-1^{l+n}→LEW; RT-1^{L}) and MHC fully mismatched donors and recipients (BN; RT-1ⁿ→LEW; RT-1^{L}), as illustrated below. Ten animals were employed in each group. The treatment protocols and surgical procedures are also described below.

| Type of Graft | Donor | Recipient |
|---|---|---|
| Isogeneic graft | Lewis (LEW; RT-1^{L}) | Lewis (LEW; RT-1^{L}) |
| Semi-allogenic | Lewis Brown Norway | Lewis (LEW; RT-1^{L}) |
| graft | (LBN; RT-1^{L+n}) | |
| Fully allogenic | Brown Norway (BN; RT-1ⁿ) | Lewis (LEW; RT-1^{L}) |
| graft | | |

I. Immunosuppressive Treatment Protocol

All recipients of different combinations of the vascularized skin and (vascularized) bone allograft (VSBA) transplants were given the immunosuppressive therapy, whereas isograft controls received no treatment. A 7-day immunosuppressive treatment protocol, similar to that described in Example 1, was employed. Briefly, the treated animal groups received 16 mg/kd/day s.c. of CsA and 250 μg/day i.p. of anti-αβ TCR mAb daily for 7 days. The first treatments were given one hour prior to transplantation. Isogeneic transplant recipients received no immunosuppressive therapy.

II. Surgical Procedures

A. Vascularized Skin Grafting Procedure

Vascularized skin allograft transplantation was performed according to the technique described by Strauch et al. (Strauch, B. and D. E. Murray. Plast. Recons. Surg. 1967; 40: 325-329). Briefly, a standard 4×6 cm template was used to mark the flap borders both in the donor and the recipient. The donor skin flap was elevated on the superficial epigastric branch of the femoral artery and vein of the donor, and end-to-end anastomoses were performed between the donor's and recipient's femoral arteries and veins using standard microsurgical techniques.

B. Vascularized Bone Transplantation

A vascularized femoral bone allograft was harvested on the femoral artery and vein of the donor, preserving supplying collateral vessels. The bone allograft was transferred to the recipient's groin region and end-to-end anastomoses between donor's and recipients femoral arteries and veins were performed using standard microsurgical techniques.

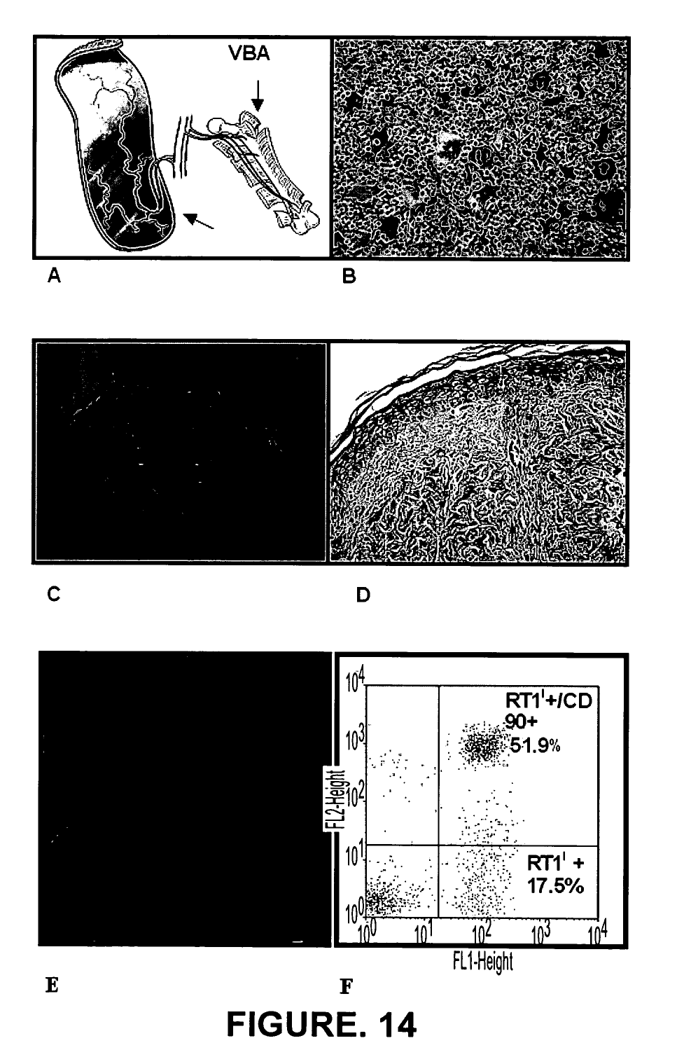

FIG. 14A is a schematic representation of the vascularized skin and bone allograft (VSBA).combining superficial epigastric skin flap and vascularized femoral bone allograft.

VSBA Transplants are Accepted Across Semi-Allogeneic and Fully-Allogeneic MHC Barriers. The VSBA isografts, and semi-allogeneic and fully allogeneic grafts in recipients receiving the combined immunosuppressive therapy, showed indefinite (over 200 days) survival of both the skin and bone components of this tissue assembly, whereas VSBA allograft controls without immunosuppressive therapy rejected uniformly within 7 days (not shown). For example, FIG. 14B illustrates a Giemsa stained (donor) vascularized bone marrow isograft 7 days after transplantation into the recipient, showing over 99% viability of the bone marrow cells in the bone transplants. FIG. 14C shows complete acceptance of a vascularized skin allograft in a representative fully allogeneic VSBA recipient transplanted across a major MHC barrier (BN→LEW) at day 63 after cessation of immunosuppression. Biopsy of the skin (hematoxylin and eosin stained) shows preserved dermis and epidermis and no histological signs of rejection (FIG. 14D).

Trafficking of Bone Marrow-Derived Cells From VSBA Transplant Recipients to Donor Bone Marrow. FIGS. 14E and 14F show immunohistostaining of frozen sections of the bone marrow tissue, and FC analysis of the bone marrow cells, respectively, taken from the donor vascularized bone transplant in the VSBA of a representative fully allogeneic recipient at day 63 after cessation of immunosuppression. The analysis showed replacement of the CD90⁺ stem cells of the donor by the recipient's CD90⁺ cells. More than 50% of the cells were CD90⁺ and RT-1^{L} (related to LEW MHC class I) positive cells of the recipient origin. These results proved the trafficking of the donor bone marrow derived stem cells between the recipient and donor bone and confirmed the viability of the transplanted vascularized bone marrow. Similar results were obtained in semi-allogeneic transplants (data not shown).

Introduction of the tolerance-inducing protocol (administration of CsA and anti-αβ T cell receptor antibody) resulted in the extension of VSBA allograft survival only in transplants receiving a bone component (over 100 days), whereas recipients of skin without bone were uniformly rejected despite CsA and anti-αβ T cell receptor antibody therapy. This was confirmed by higher levels of mixed chimerism in skin/bone allograft recipients, whereas recipients of skin without bone showed only trace levels of chimerism.

### Example 11

Development of a Trimeric Recipient from Fully Allogeneic Transplants from Genetically Unrelated Donors

The VSBA transplantation surgical procedure was performed as described in Example 4. Each Lewis (LEW, RT-1^{L}) rat received a vascularized bone allograft, including a vascularized skin flap, from two genetically unrelated allograft donors, i.e., Brown Norway (BN, RT-1ⁿ) and ACI (A x C Irish, RT-1^{a}) rats. Both VSBA transplantations were performed during the same operative procedure.

Control LEW recipients received a vascularized skin allograft alone, without the vascularized bone component, from both fully allogeneic donors.

All LEW recipient received a 7-day immunosuppressive treatment protocol, described in Example 1. Briefly, the treated animal groups received 16 mg/kd/day s.c. of CsA and 250 μg/day i.p. of anti-αβ TCR mAb daily for 7 days. The initial treatments were given at the time of transplantation at the time of clamp release

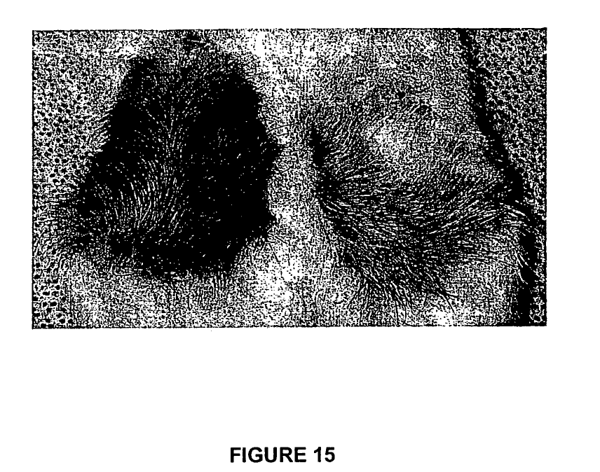

Two Genetically Unrelated VSBA Transplants are Accepted Across Fully-Allogeneic MHC Barriers, Producing a Fully Tolerant Trimeric Recipient. FIG. 15 illustrates an LEW recipient of two genetically unrelated VSBA transplants at day 35 after transplantation: the vascularized bone transplants are not visible, as they are beneath the transplanted skin flaps shown. On the left is a VSBA transplant from a BN donor, showing full skin acceptance by the LEW recipient of this fully allogeneic transplant. On the right is a VSBA transplant from an ACI donor, showing full skin acceptance by the LEW recipient of this fully allogeneic transplant.

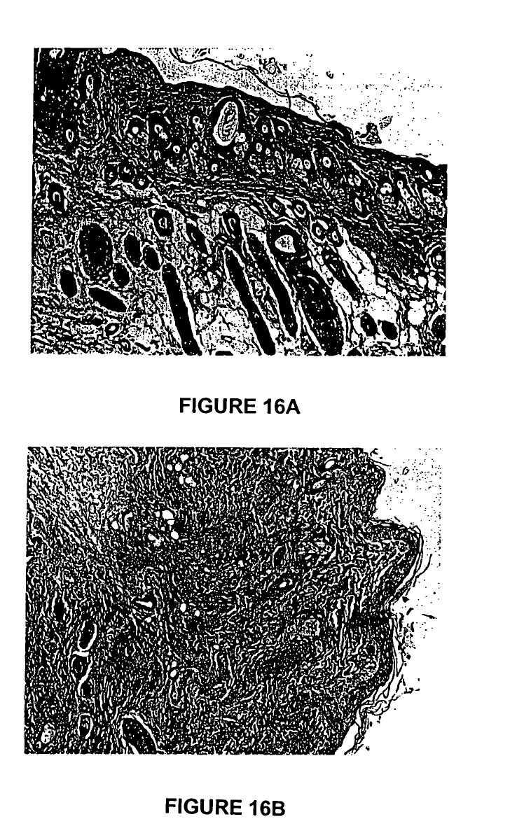

FIGS. 16A and 16B illustrate H&E stained formalin-fixed skin tissues taken from the BN allograft and the ACI allograft, respectively, at day 21 after transplantation, showed preserved dermis and epidermis and no histological signs of rejection. At over 100 days after transplantation (to the present), neither of the skin grafts show any signs of rejection.

Determination of the Donor Specific Trimerism. Flow cytometry analysis was performed on PBMC of the LEW recipients at day 21 and day 35 after transplantation of the VSBAS transplants from the BN and ACI donors.

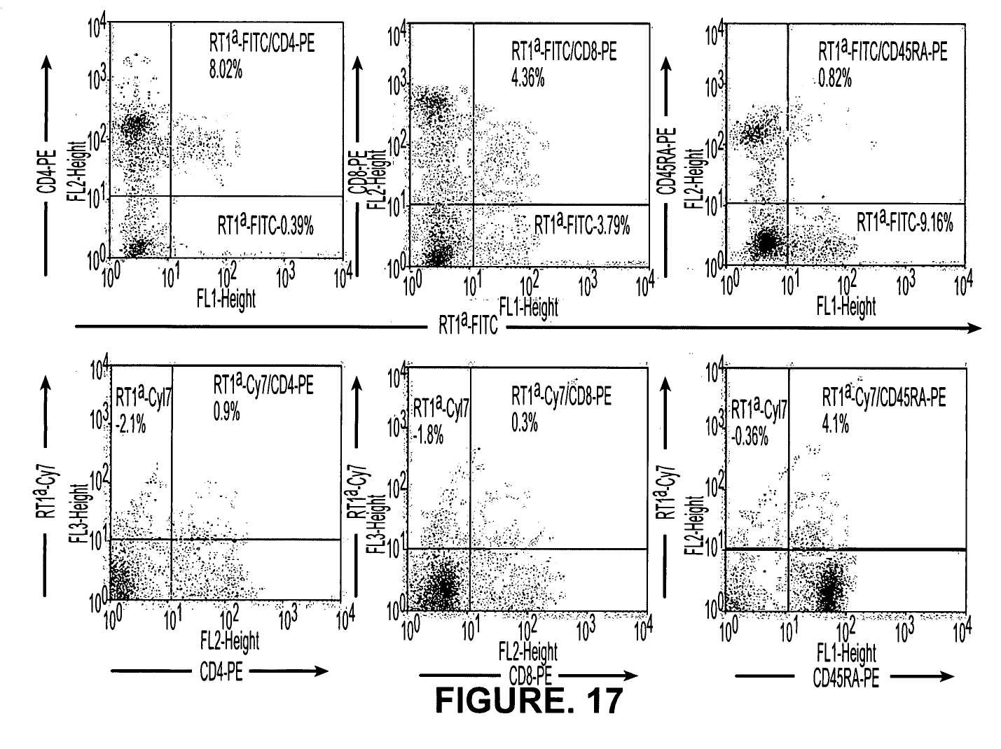

The dot-plot results of the lymphoid cell subpopulations obtained from quadrangle analytical gates demonstrated the presence of double positive RT-1^{a}-FITC/CD4⁻PE, RT-1^{a}-FITC/CD8⁻PE and RT-1^{a}-FITC/CD45RA⁻PE (ACI/LEW) at a level of 8.02%, 4.36% and 0.82%, respectively, of PBMC on day 21; and also the presence of double positive RT-1ⁿ-Cy7/CD4⁻PE, RT-1ⁿ-Cy7/CD8⁻PE and RT-1ⁿ-Cy7/CD45RA⁻PE (BN/LEW) at a level of 0.9%, 0.3% and 4.1%, respectively (FIG. 17, bottom horizontal row). The expression of RT-1^{a} (RT-1^{a}-FITC) antigen and RT-1ⁿ (RT-1ⁿ-Cy7) antigen (FIG. 11, top horizontal row and bottom horizontal row, respectively) on non-CD4, non-CD8, and non-CD45RA positive T cell populations suggests the existence of donor/recipient trimeric residents not evaluated with the PE-conjugated mAb against surface specific antigen.

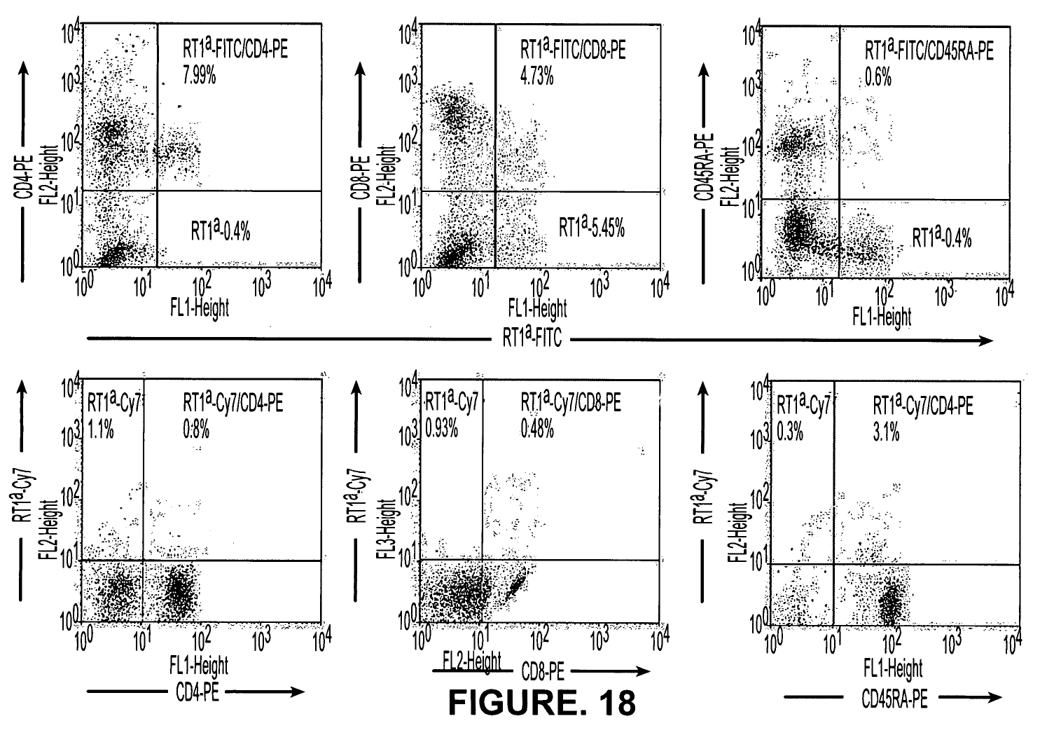

Dot plot results obtained from the peripheral blood of LEW recipients on day 35 are illustrated in FIG. 12. The results demonstrated the presence of double positive RT-1^{a}-FITC/CD4⁻PE, RT-1^{a}-FITC/CD8⁻PE and RT-1^{a}-FITC/CD45RA⁻PE (ACI/LEW) at a level of 7.99%, 4.73% and 0.6%, respectively, of PBMC on day 35 (FIG. 18, top horizontal row); and also the presence of double positive RT-1ⁿ-Cu7/CD4⁻PE, RT-1ⁿ-Cy7/CD8⁻PE and RT-1ⁿ-Cy7/CD45RA⁻PE (BN/LEW) at a level of 0.8%, 0.48% and 3.1%, respectively (FIG. 18, bottom horizontal row).

These results show that the trimerism obtained in these LEW recipients was stable and correlated with the maintenance of tolerance to both VSBA fully allogeneic allografts.

In contrast, control LEW recipients receiving vascularized skin flaps without vascularized bone transplants showed a transient chimerism (less than 1% to 1%) that declined over time leading to allograft rejection within 40 days from the time of transplantation (data not shown).

Therefore, this example again illustrates the tolerance-inducing and chimerism/trimerism inducing properties of the vascularized bone component of the transplant. It has further been demonstrated that donor-specific cells can be produced in the recipient and can co-exist without rejection in the recipient. No recipient preconditioning is required.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

### Example 12

Purification of CD90⁺ Cells

Bone marrow cells are labeled with monoclonal antibody CD90+. Labeled cells are conjugated with anti-FITC MACS MicroBeads. After magnetic labeling, the cells are passed through a separation column, which is placed in a strong permanent magnet. The magnetically labeled cells are retained in the column and separated from the unlabeled cells, which pass through. After removing the column from the magnetic field, the retained fraction can be eluted. This fraction contains CD90+ cells.
1. Bone marrow cells flushed from femur and tibia bone into Medium 199 containing 2 μg/ml gentamycin.
2. Count the cells and resuspend 1×10⁷ in wash buffer (0.5% BSA+2mMEDTA in Ca and Mg free PBS).
3. After centrifugation resuspend the pellet and add CD90 MoAb (100 μl/1×10⁷ cells) and incubate 30 min at 6-12° C. Wash the cells in wash buffer.
4. Add anti-FITC Micro Beads to cell pellet (20 μl/1×10⁷ cells) and incubate 30 min at 6-12° C. Wash cells and resuspend in 500 μl wash buffer.
5. Place MACS separation column in the MiniMACS magnet. Fill the column wash buffer.
6. Apply the magnetically labeled cells onto the column. The negative cells (CD90−) pass through the column.
7. Remove MACS separation column from the magnet and place it on a Falcon tube.
8. Apply wash buffer onto the column and flush out positive fraction (CD90+) with gentle pressure.
   For increased purity steps 5-8 may be repeated using new MACS separation column.

1. A method for inducing donor-specific tolerance and/or mixed donor-recipient chimerism in an allograft transplant recipient, comprising the steps of:
a) administering to said recipient a therapeutically effective amount of an immunosuppressive agent that depletes T cells;
b) administering to said recipient a therapeutically effective amount of anti-T cell receptor antibodies; and
c) administering to said recipient a therapeutically effective amount of donor stem cells by intraosseous delivery;
wherein said immunosuppressive agent, said antibodies and said stem cells induce donor-specific tolerance and/or mixed donor-recipient chimerism in said recipient.

2. The method of claim 1, wherein said donor stem cells are contained within donor bone marrow.

3. The method of claim 1, wherein said donor stem cells are isolated from donor bone marrow.

4. The method of claim 3, wherein said donor stem cells are seeded into a bone scaffold.

5. The method of claim 4, wherein said bone scaffold is implantable.

6. The method of claim 3, wherein said donor hematopoietic stem cells are expanded and propagated in said bone scaffold.

7. The method of claim 1, wherein the therapeutically effective amount of allograft donor bone marrow cells is an amount sufficient to induce the production of mixed donor-recipient chimerism in the recipient.

8. The method of claim 7, wherein the mixed donor-recipient chimerism in the recipient comprises a level of mixed donor and recipient cells of about 5% to about 50% of circulating peripheral blood mononuclear cells.

9. The method of claim 8, wherein the level of mixed donor and recipient cells is about 10% to about 40% of circulating peripheral blood mononuclear cells.

10. The method of claim 9, wherein the level of mixed donor and recipient cells is about 20% to about 30% of circulating peripheral blood mononuclear cells.

11. The method of claim 1, wherein the therapeutically effective amount of the allograft donor bone marrow cells is an amount sufficient to maintain long-term recipient tolerance of the allograft without readministration of the immunosuppressive agent and the anti-αβ T cell receptor antibodies.

12. The method of claim 1, wherein the step of administering hematopoietic stem cells comprises the substeps of implanting and vascularizing an allograft donor bone including the bone marrow into the recipient.

13. The method of claim 12, wherein the allograft donor bone further includes an attached portion of donor skin, and wherein the substeps of implanting and vascularizing the donor bone into the recipient further includes the substeps of implanting and vascularizing the attached portion of donor skin.

14. The method of claim 1, wherein the step of administering hematopoietic stem cells comprises the substeps of obtaining crude bone marrow from a bone of the allograft donor, and implanting the donor crude bone marrow into a bone marrow cavity of the allograft recipient.

15. The method of claim 14, wherein the substep of implanting the allograft donor crude bone marrow further comprises the substeps of removing a quantity of crude bone marrow from a recipient bone marrow compartment to provide a space in the compartment, and directly implanting the allograft donor crude bone marrow into the space.

16. The method of claim 1, wherein the step of administering hematopoietic stem cells comprises the substeps of obtaining a bone marrow nucleated cell suspension from a bone of the allograft donor, and implanting the bone marrow nucleated cell suspension into the recipient by intraosseous delivery.

17. The method of claim 1, wherein the step of administering hematopoietic stem cells comprises the substeps of obtaining a bone marrow cell suspension from a bone of the allograft donor; isolating a nucleated subpopulation of the bone marrow cells, and implanting the nucleated subpopulation of the bone marrow cells into the recipient by intraosseous delivery.

18. The method of claim 17, wherein said subpopulation of the isolated bone marrow cells includes tolerance inducing cells.

19. The method of claim 17, wherein the subpopulation of the isolated bone marrow cells includes CD4⁺ cells.

20. The method of claim 17, wherein the subpopulation of the isolated bone marrow cells includes CD8⁺ cells.

21. The method of claim 17, wherein the subpopulation of the isolated bone marrow cells includes B cells.

22. The method of claim 1, wherein the allograft is selected from the group consisting of composite tissue, solid organs, glands, glandular cells, skin, cartilage, hematopoietic tissue, lymphoid tissue, tendons, ligaments, muscles, nerve tissue, vascular tissue and combinations thereof.

23. A system for inducing donor-specific tolerance and/or mixed donor-recipient chimerism in an allograft transplant recipient, comprising: (a) a combination of pharmaceutical compositions for depletion of T cells in a recipient of an allograft from a donor, comprising an effective amount of a pharmaceutical composition that comprises an immunosuppressive T cell-depleting agent, and an effective amount of a pharmaceutical composition that comprises anti-T cell receptor antibodies, wherein administration of the combination to the recipient results in elimination of about 50% to about 99.9% of T cells circulating in peripheral blood of the recipient; (b) a delivery system for implanting a therapeutically effective amount of hematopoietic stem cells from the allograft donor into the allograft recipient, wherein the delivery system is selected from the group consisting of an implantable vascularizable bone from the allograft donor including the donor bone marrow; implantable crude donor bone marrow; an implantable donor bone marrow nucleated cell suspension; an implantable isolated subpopulation of donor bone marrow cells; an artificial bone scaffold seeded with hematopoietic stem cells; and combinations thereof.

24. The system of claim 23 wherein the implantable isolated subpopulation of bone marrow cells is selected from the group consisting of tolerance inducing cells, hematopoietic stem cells, hematopoietic progenitor cells, CD4+ cells, CD8+ cells, B-cells, CD34⁺ cells and combinations thereof.

25. The system of claim 23 wherein the effective amount of the combination of pharmaceutical compositions is sufficient to eliminate about 75% to about 95% of circulating T cells.

26. The system of claim 25 wherein the effective amount of the combination is sufficient to eliminate about 80% to about 90% of circulating T cells.

27. The system of claim 26, wherein the immunosuppressive agent comprises cyclosporine A.

28. The system of claim 23, wherein the recipient is a human.

29. A method for maintaining a level of mixed donor-recipient chimerism in an allograft transplant recipient, comprising the steps of: (a) inducing donor-specific tolerance and/or mixed donor-recipient chimerism in an allograft recipient by (i) administering to a recipient of an allograft a therapeutically effective amount of an immunosuppressive agent that depletes T cells; (ii) administering to the recipient of the allograft a therapeutically effective amount of anti-T cell receptor antibodies; (iii) implanting an allograft from an allograft donor into the recipient; and (iv) implanting a therapeutically effective amount of hematopoietic stem cells from the allograft donor into the allograft recipient by intraosseous delivery; (b) determining an optimal level of chimeric cells in the recipient not undergoing rejection of the allograft; (c) harvesting chimeric cells from the recipient when the optimal level of chimeric cells is achieved; (d) reconstituting the recipient with the harvested chimeric cells when the level of chimeric cells in the recipient falls below a minimum level of chimeric cells; and (e) optionally readministering an effective amount of the immunosuppressive agent and/or the anti-T cell receptor antibodies and/or stem cells and/or chimeric cells.

30. The method of claim 29, wherein the step of reconstituting the recipient comprises administering the chimeric cells by a method selected from the group consisting of direct intraosseous injection into a recipient bone marrow cavity; direct intraosseous implantation into a bone marrow cavity of an implanted donor bone allograft; seeding into artificial bone marrow scaffold followed by implantation of said artificial bone marrow scaffold; intravenous injection into the recipient; and combinations thereof.

31. The method of claim 29, wherein the minimum level of chimeric cells is about 5% to about 20% of circulating peripheral blood mononuclear cells.

32. The method of claim 29, wherein the optimal level of chimeric cells is about 10% to about 50% of circulating peripheral blood mononuclear cells.

33. The method of claim 30, wherein the optimal level of chimeric cells is about 20% to about 30% of circulating peripheral blood mononuclear cells.

34. The method of claim 29, wherein the harvesting step comprises isolating chimeric cells from the peripheral blood of the recipient.

35. A method for transplanting stem cells from a donor to a recipient comprising administering bone marrow from said donor into a bone of a recipient such that said recipient becomes chimeric for at least one of T cells and B cells.

36. The method of claim 35, wherein said stem cells are hematopoietic stem cells.

37. The method of claim 36, wherein said hematopoietic stem cells are CD34⁺ cells.

38. The method of claim 35, wherein said bone marrow is unprocessed bone marrow.

39. The method of claim 35, wherein said bone marrow is a crude bone marrow preparation in a form suitable for intraosseous implantation.

40. The method of claim 35, wherein said bone marrow is artificial bone marrow or a bone marrow substitute that has been seeded with donor stem cells prior to transplantation.

41. The method of claim 35, wherein said donor and said recipient are of the same species.

42. The method of claim 35, wherein said donor and said recipient share a plurality of HLA markers.

43. The method of claim 35, wherein said donor and said recipient share no HLA markers.

44. The method of claim 35, wherein said donor and said recipient share a single HLA marker.

45. The method of claim 35, wherein said bone marrow is transplanted to said recipient in combination with an immunosuppressant, an anti-αβ T cell receptor antibody, or a combination of an immunosuppressant and an anti-αβ T cell receptor antibody.

46. The method of claim 43, wherein said bone marrow is transplanted as an adjunct to the transplantation of an allograft.

47. The method of claim 44, wherein said allograft is a composite tissue allograft.

48. A method of monitoring the status of a primary allograft in a recipient, wherein said recipient has received a monitor allograft, comprising:
(a) examining the monitor allograft for a plurality of days after transplantation of said primary allograft and said monitor allograft;
(b) determining on each of said plurality of days whether the monitor allograft exhibits one or more signs of rejection or graft versus host disease; and
(c) if monitor allograft exhibits one or more signs of rejection or graft versus host disease, administering an immunosuppressant, an anti-T cell receptor antibody and a plurality of bone marrow cells derived from the donor of said allograft, in an amount sufficient to reduce said signs of rejection or graft versus host disease.

49. The method of claim 48, wherein said plurality of bone marrow cells are contained within unprocessed bone marrow or a crude bone marrow suspension.

50. The method of claims 1, 23 or 29, wherein said anti-T cell antibody is an anti-αβ T cell receptor antibody.
